(19) United States
(12) Patent Application Publication
(10) Pub. No.: US 2018/0179488 A1
(43) Pub. Date: Jun. 28, 2018

(54) METHOD FOR RESTORING SENSITIVITY OF ANTIBIOTIC RESISTANT BACTERIA TO ANTIBIOTICS AND REDUCING THE EXPRESSION OF THE HILA GENE

(71) Applicant: Diamond V Mills, Incorporated, Cedar Rapids, IA (US)

(72) Inventors: Victor Leonard NSEREKO, Johnston, IA (US); Mark F. SCOTT, Cedar Rapids, IA (US); Donald R. McINTYRE, Pageland, SC (US); Steve CARLSON, Ames, IA (US); Darin Lee HENRY, Camas, WA (US)

(21) Appl. No.: 15/852,634

(22) Filed: Dec. 22, 2017

Related U.S. Application Data
(60) Provisional application No. 62/438,053, filed on Dec. 22, 2016.

(51) Int. Cl. C12N 1/20; A61K 35/74; A23K 50/70; A23K 50/75; A23K 50/10; A23K 20/142; A23K 20/147; A23K 20/158; A23K 20/22; A23K 20/26; A23K 20/24

(52) U.S. Cl. CPC C12N 1/20; A61K 35/74; A23K 50/70; A23K 50/75; A23K 50/10; A23K 20/24; A23K 20/147; A23K 20/158; A23K 20/22; A23K 20/26; A23K 20/142

(57) ABSTRACT

A method for restoring the sensitivity of antibiotic resistant bacteria to antibiotics and a method for reducing the expression of hilA gene in animals. In particular, the method includes the step of introducing a fermented product into the diet of an animal. Certain components of the fermented product interact with the bacteria within the animal such that the method produces the beneficial effects of reducing the expression of the hilA gene and also restores the sensitivity of antibiotic resistant bacteria to antibiotics.

### CROSS-REFERENCE TO RELATED APPLICATIONS

The present application claims priority to provisional patent application 62/438,053 which was filed on Dec. 22, 2016, and is hereby expressly incorporated by reference in its entirety.

### BACKGROUND OF THE INVENTION

Antibiotics acre medicines used to prevent and treat bacterial infections. Antibiotic resistance occurs when bacteria change in response to the use of these medicines. The bacteria become antibiotic resistant. When the resistant bacteria infect humans or animals, the infections they cause are much harder treat. Antibiotic resistance is rising to dangerously high levels in all parts of the world (World Health Organization (WHO) fact sheet, 2017). New resistance mechanisms are emerging and spreading globally, threatening our ability to treat common infectious diseases. A growing list of infections—such as foodborne diseases—are becoming harder, and sometimes impossible, to treat as antibiotics become less effective (WHO fact sheet, 2017). The present invention relates to a method for restoring the sensitivity of antibiotic resistant bacteria to antibiotics and a method for reducing the expression of the hilA gene. A fermented product is introduced into the diet of an animal including humans.

It is an object of the invention to provide a method that can restore the sensitivity of antibiotic resistant bacteria to antibiotics.

It is further an object of the invention to provide a method to reduce the expression of the hilA gene.

### SUMMARY OF THE INVENTION

The present invention is a method for restoring the sensitivity of antibiotic resistant bacteria to antibiotics and a method for reducing the expression of the hilA gene. A fermented product is introduced into the diet of an animal. The fermented product contains one or more ingredients that have the beneficial effects noted above.

### DESCRIPTION OF THE FIGURES

FIG. 1 shows Salmonella fecal shedding in broilers fed a fermented product versus a non-fermented product.

FIG. 2 shows the prevalence of Salmonella fecal shedding in broilers.

FIG. 3 shows intestinal colonization by Salmonella in broilers.

FIG. 4 shows prevalence of intestinal colonization by Salmonella in broilers.

FIG. 5 shows tissue culture invasiveness of Salmonella recovered from broilers.

FIG. 6 shows expression of hilA in Salmonella recovered from feces of broilers.

FIG. 7 shows chloramphenicol resistance of Salmonella recovered from the feces or intestines of broilers.

FIG. 8 shows the presence of SG11 in Salmonella recovered from the feces or intestines of broilers.

FIG. 9 shows Salmonella fecal load in heifers fed a fermented product versus a non-fermented product.

FIG. 10 shows the prevalence of Salmonella fecal shedding in heifers.

FIG. 11 shows the assessment of lymph node infiltration by Salmonella in heifers.

FIG. 12 shows the assessment of the prevalence of Salmonella lymph node infiltration in heifers.

FIG. 13 shows E. coli fecal load in heifers.

FIG. 14 shows the prevalence of E. coli fecal shedding in heifers.

FIG. 15 shows the tissue culture invasiveness of Salmonella recovered from the feces of lymph nodes of heifers.

FIG. 16 shows the semi-quantitation of hilA expression of Salmonella recovered from the feces and lymph nodes of heifers.

FIG. 17 shows the prevalence of antibiotic resistant Salmonella recovered from the feces and lymph nodes of heifers.

FIG. 18 shows the prevalence of certain Salmonella serotypes recovered from the feces and lymph nodes of heifers.

FIG. 19 shows the influence of XPC on growth of Salmonella in broilers.

FIG. 20 shows the effect of XPC on invasiveness of Salmonella in broilers.

FIG. 21 shows the effect of XPC on antibiotic resistance of Salmonella in broilers.

FIG. 22 shows the effect of XPC on expression of hilA in Salmonella from an in vitro model assay.

FIG. 23 shows prevalence and numbers of Salmonella in cows.

FIG. 24 shows a dot plot of Salmonella numbers in cows.

FIG. 25 shows the virulence of Salmonella recovered in cows.

FIG. 26 shows the virulence of Salmonella as measured by the expression of hilA in cows.

FIG. 27 shows the antibiotic resistance of Salmonella in cows.

FIG. 28 shows the prevalence and numbers of E. coli in cows.

FIG. 29 shows a dot plot of E. coli numbers in cows.

FIG. 30 shows the antibiotic resistance of E. coli in cows.

FIG. 31 shows the prevalence of Salmonella in poultry.

FIG. 32 shows the numbers of Salmonella in poultry.

FIG. 33 shows the prevalence of Salmonella utilizing cloaca and environmental swabs.

FIG. 34 shows the numbers of Salmonella utilizing cloaca and environmental swabs.

FIG. 35 shows the prevalence and numbers of Salmonella in broilers.

FIG. 36 shows the total Salmonella load to plant in broilers.

FIG. 37 shows a dot plot of Salmonella numbers in broilers.

FIG. 38 shows Salmonella prevalence and numbers in broilers.

FIG. 39 shows Salmonella prevalence and numbers in turkeys.

FIG. 40 shows Salmonella load to plant in turkeys.

FIG. 41 shows a dot plot of Salmonella numbers in turkeys.

FIG. 42 shows the prevalence and numbers of Salmonella in turkeys.

FIG. 43 shows the prevalence and numbers of Salmonella utilizing cloaca swabs.

FIG. 44 shows a dot plot of Salmonella numbers utilizing cloaca swabs.

FIG. 45 shows the virulence of Salmonella in broilers.

FIG. 46 shows the virulence of Salmonella in turkeys.

FIG. 47 shows the virulence of Salmonella recovered from layer Cloaca swabs.

FIG. 48 shows the reduction of virulence of Salmonella in broilers.

FIG. 49 shows the reduction of virulence of Salmonella in turkeys.

FIG. 50 shows the reduction of virulence of Salmonella recovered from layer Cloaca swabs.

FIG. 51 shows the antibiotic resistance of Salmonella in broilers.

FIG. 52 shows the antibiotic resistance of Salmonella in broilers.

FIG. 53 shows the antibiotic resistance of Salmonella in turkeys.

FIG. 54 shows the antibiotic resistance of Salmonella in turkeys.

FIG. 55 shows the antibiotic resistance of Salmonella recovered from layer Cloaca swabs.

FIG. 56 shows the antibiotic resistance of Salmonella in broilers.

FIG. 57 shows the antibiotic resistance of Salmonella recovered from Cloaca swabs.

FIG. 58 shows the antibiotic resistance of Salmonella in broilers.

FIG. 59 shows the antibiotic resistance of Salmonella recovered from Cloaca swabs.

FIG. 60 shows the antibiotic resistance of Salmonella in broilers.

FIG. 61 shows the antibiotic resistance of Salmonella recovered from Cloaca swabs.

FIG. 62 shows the antibiotic resistance of Salmonella in broilers.

FIG. 63 shows the antibiotic resistance of Salmonella recovered from Cloaca swabs.

FIG. 64 shows a survey of broiler E. coli APEC genotyping.

FIG. 65 shows a survey of turkey E. coli APEC genotyping.

FIG. 66 shows a survey of broiler E. coli antibiotic resistance.

FIG. 67 shows a survey of turkey E. coli antibiotic resistance.

### DETAILED DESCRIPTION OF THE INVENTION

The inventive process introduces a fermented product to the diet of animals. The optimal dose may vary depending on the particular animal to be treated. The fermented product is preferably derived from a natural media that has been treated with at least one type of yeast. The yeast at least partially consume the media, and due to certain biochemical reactions, produce functional metabolites. The fermented product is then added to the feed to achieve a particular ratio of fermented product to total feed. The preferred range is the fermented product comprising between 0.004% and 0.2% of the total feed, with an ideal percentage of 0.125% for poultry and 0.2% for other monogastric animals. For ruminant animals, the preferred range is the fermented product comprising a proportion of feed such that the animal receives 2 to 30 g per day, with an ideal feeding rate of 19 g per head per day. If the animal is a human, then the ingested amount is 500 mg per day.

Original XPC™, NutriTek™ and NaturSafe™ from Diamond V (hereinafter “XPC”, “NutriTek” and “NaturSafe”) contains functional metabolites produced during a fermentation process by the yeast Saccharomyces cerevisiae. The XPC can be utilized as the fermented product in the process described herein. When added to the feed of poultry, XPC has been shown to reduce the colonization and shedding of pre-harvest foodborne pathogens. Similarly, when fed to cattle, both NaturSafe and NutriTek have been shown to reduce colonization and shedding of pre-harvest foodborne pathogens. NaturSafe, NutriTek and XPC fall under “yeast culture” in the 2015 Official Publication of the Association of American Feed Control Officials. The ideal characteristics of XPC include: crude protein is a minimum of 12.0%, crude fat is a minimum of 1.2%, crude fiber is a maximum of 30.0%, and ash is a maximum of 10.8%, together with at least fourteen amino acids at varying levels greater than 0.16% such as leucine, proline, glycine, and valine, and also a diverse group of minerals that includes potassium, phosphorus, and calcium. The ideal characteristics of NaturSafe and NutriTek include: crude protein is a minimum of 18.0%, crude fat is a minimum of 1.2%, crude fiber is a maximum of 20.0%, and ash is a maximum of 13%, together with at least fourteen amino acids at varying levels greater than 0.12% such as leucine, proline, glycine, and valine, and also a diverse group of minerals that includes potassium, phosphorus, and calcium.

Studies have demonstrated that feeding XPC suppresses pathogen prevalence and load. This discovery is the subject of the first patent. In the case of Salmonella in poultry for example, the result is that we have less of it available in the gut to contaminate the meat during processing. This lower level of contamination makes other additional post-harvest interventions (intended to cut back on contamination) more effective since they have less Salmonella to work on at the onset. Ultimately, the meat on the grocery shelf has a much-reduced level of contamination and is less likely to make the public sick. We know that if we add Salmonella to a tube containing XPC, chicken feed and mixed gut microorganisms obtained from chickens feces, XPC reduces the growth of Salmonella such that there is less Salmonella present at the end of the incubation. We also know that if we don't have the mixed gut microorganisms present, XPC does not have these effects on Salmonella growth. Therefore, this effect can be demonstrated outside the animal.

We also now know that feeding XPC to chickens that have been challenged with Salmonella makes the Salmonella recovered from those chickens less invasive or virulent, as determined in experiments that utilize human epithelial cells to determine invasiveness. What this means basically is that the live Salmonella that is still present in chickens fed XPC is less dangerous to humans that may ingest it when it contaminates the meat they subsequently eat. Additionally, we have been able to demonstrate, multiple times, that feeding XPC down regulates the hilA gene which is believed to be the master regulator of virulence. Hence in addition to load and prevalence, as discussed in (1), XPC makes Salmonella less likely to result in illness, following ingestion, by down-regulating the expression of the gene that is involved in the onset of illness. Further, as in 1; this effect has been demonstrated in tubes in the lab and is also dependent of the presence of mixed gut microorganisms.

Our studies have demonstrated another effect that we believe is the most important for public health. We have demonstrated multiple times that feeding XPC to birds challenged with a Salmonella Serovar known to be resistant to multiple antibiotics, forced the Salmonella to revert to being sensitive to the antibiotics. Specifically, chickens were challenged with a Salmonella Typhimurium isolate containing a mobile genetic element (SGI1) that possesses genes that confer resistance to five antibiotics. We demonstrated that feeding XPC made the Salmonella expel the genetic element, rendering it susceptible to the antibiotics again. This effect has now been demonstrated in a number of animal species, on Salmonella and E. coli, and with different antibiotics for which resistance is spread by a variety of differing mechanisms. Whereas we demonstrated that the SGI1 integron was expelled in chickens, on feeding XPC, it is likely that other mobile genetic elements that transfer antibiotic resistance are similarly affected. Further, again, we have demonstrated that this effect can occur in tubes in the lab and is dependent of the presence of mixed gut microorganisms.

### Example 1

### Effects of Feeding XPC a Saccharomyces cerevisiae Fermentation Product, on Virulence, Antibiotic Resistance, and Fecal Shedding of Salmonella Typhimurium in Broilers (Feye et al. Poult Sci. 2016 Dec. 1; 95 (12):2902-2910)

A controlled experiment was conducted using one-day-old broiler chicks. Three separate and independent replications of this experiment were conducted using a total of fifty chicks per replicate (25 per treatment group; 75 chicks per treatment). On day zero (DO), birds were housed in a BL-2 facility in pens (0.09 m2; 10 birds/pen) within rooms that were both humidity (˜40%) and temperature controlled (35° C. for 3 d, then 28 to 31° C. for the remainder of the study). On D14, birds were moved to elevated Tenderfoot-type decks (13.4 m2 per treatment group) for the remainder of each experiment. Feed was provided in a metal feed trough and water through a bell drinker.

All birds were fed a non-medicated starter diet (24% crude protein) from DO to 21. Birds were then randomly assigned on D21 to one of 2 feed treatment groups: 1) finisher control diet only (CON), or 2) finisher diet that contained 1.25 kg/MT XPC. From D21 to 49, the basal diet was a non-medicated finisher diet (18 to 19% crude protein) and birds were allowed ad libitum access to feed and water. The photoperiod consisted of 12 h light and 12 h dark. All birds were individually weighed on D21 and then again at the end of the study on D49.

Each room held one treatment group to avoid inadvertently administering the wrong treatment within a room. Throughout the three consecutive studies, treatment groups were alternated in the two different rooms to avoid a potential room effect. The investigators were blinded as to which birds received the CON or XPC diet during the entire study.

All birds were confirmed to be Salmonella-free by fecal culture upon arrival. Specifically, one to 5 g of freshly voided feces from each chick was diluted in 10 mL of Lennox L broth. After settling for one to 2 h at room temperature, an aliquot (100 μL) of this mixture was streaked onto and then incubated overnight at 37° C. on Xylose Lysine Deoxycholate (XLD) agar that is selective for Salmonella, which appear as white colonies with black centers. All pre-infection fecal samples were free of Salmonella.

On D2, 9, and 16, birds were orally inoculated with Salmonella Typhimurium strain LNWI. The dose increased from 2×108 CFU/bird on D2 to 4×108 CFU/bird on D9 to 8×108 CFU/bird on D16, and this procedure was done to maximize the likelihood of large intestinal carriage. The Salmonella inoculum was prepared and dosed as previously reported. The inoculum was slowly introduced into the mouth of each bird using a pipette tip. Previous studies revealed that Salmonella is viable after incubation with either XPC (at the concentration equivalent to the dose used in this study) or the CON treatment.

On D6, 13, and 20, one to 5 g of freshly voided feces from each bird was diluted in 10 mL of Lennox L broth. After settling for one to 2 h at room temperature, an aliquot (100 μL) of this mixture was streaked onto and then incubated overnight at 37° C. on XLD agar. On D7 and 14, fecal samples were examined for the qualitative presence of Salmonella colonies on XLD agar. On D21, Salmonella were enumerated on XLD agar and shedding was determined quantitatively as number of colonies×100 (i.e., the dilution factor) divided by the grams of feces in the sample. Any non-shedding individual birds (as determined by fecal culture, n=6 per group per each of the 3 separate trials) were euthanized and removed from the study on D21. The remaining birds were assigned to either treatment group based on body weight and Salmonella shedding, using a serpentine assignment format that mathematically redistributes birds in order to prevent a weight bias between groups. Specifically, each bird was ranked based on weight and the bird with the lowest weight was grouped (e.g., Treatment Group A) with the bird with the highest weight; the bird with the second lowest weight was placed in the other group (Treatment Group B) along with the bird with the second highest weight; the bird with the third lowest weight was placed in Group A along with the bird with the third highest weight; the bird with the fourth lowest weight was placed in Group B along with the bird with the fourth highest weight, and so forth. As an illustrative example using 36 birds segregated into 2 treatment groups (either XPC or CON), the following body weight-based rankings would be used in each group: Treatment Group A, birds 1, 36, 3, 34, 5, 32, 7, 30, 9, 28, 11, 26, 13, 24, 15, 22, 17, and 21; Treatment Group B, birds 2, 35, 4, 33, 6, 31, 8, 29, 10, 27, 12, 25, 14, 23, 16, 21, 18, and 19. Fecal shedding was also factored into the assignments for birds with identical weights or when an odd number of birds was available for segregation into the 2 groups. That is, the fecal shedding data were considered, when necessary, in order to make the average fecal shedding equivalent between the groups.

On D21, treatments began for each group of birds (n=19 to 22 per group after removing non-shedders in each experiment). On D28, 35, and 42, approximately 0.5 g of freshly voided feces (from each bird) was briefly vortexed in 10 mL of Lennox L broth. An aliquot of this mixture (100 μL) was incubated overnight at 37° C. on XLD agar. The following d, white colonies with black centers were enumerated and CFU/g of feces was calculated based on a dilution factor equal to 100.

On D49, all remaining birds were euthanized and a 5 cm section (approximately 3 g) of distal intestine (between the cloaca and ceca) was aseptically removed from each bird and cut longitudinally. Each section was placed in 10 mL Lennox L broth and briefly vortexed to dislodge the Salmonella. An aliquot (100 μL) of this mixture was then dispersed onto XLD agar plates that were incubated overnight at 37° C. The following d, white colonies with black centers were enumerated and CFU/g of intestine was calculated based on a dilution factor equal to 100.

On D21, 28, 35, 42, and 49, Salmonella recovered from broiler chickens were subjected to a mammalian tissue culture invasion assay. After enumeration of colonies on XLD agar plates, approximately 30% of recovered colonies were immediately inoculated en masse into LB broth that was used in a standard gentamicin protection-based invasion assay using Human Epithelial Type 2 cells, with a multiplicity of infection equal to at least one. Bacteria were allowed to adhere and invade tissue culture cells for one h, after which the extracellular (i.e., non-invasive) were killed with 50 μg/ml gentamicin. Tissue culture cells were then lysed with 1% Triton and the lysates were plated on XLD agar and grown overnight at 37° C. The next d, colonies were counted and percent invasion was calculated as 100×(number of Salmonella recovered from tissue culture wells/number of Salmonella incubated with tissue culture wells). Invasion assays were performed in triplicate for both groups (XPC and CON) in each of the 3 separate experiments. Bacteria isolated from XLD were done so immediately to prevent changes in invasion gene expression.

Approximately 20% of Salmonella recovered from the birds were subjected to a semi-quantitative RT-PCR that assesses the expression of hilA the global regulator of Salmonella invasion. RNA was isolated and subjected to the semi-quantitative RT-PCR assay in which the number of PCR cycles (5 to 40) required to visualize an amplicon on agarose gel electrophoresis is documented. RNA was isolated from a group of colonies (n>40 colonies) picked directly from XLD plates and placed into PBS. RNA was isolated using the RNEasy kit as per the manufacturer's protocol.

RNA (50 ng/assay) was subjected to the semi-quantitative RT-PCR assay in which the number of PCR cycles (5 to 40) required to visualize an amplicon on agarose gel electrophoresis is documented. PCR conditions and the hilA primers are described previously (Carlson et al, Infect. Immun. 2007; 72:792-800). The rpoS primers are 5′-ATGAGTCAGAATACGCTGAA-3′ and 5′-TTACTCGCGGAACAGCGCTT-3′, representing the forward and reverse primers, respectively.

Subsets of reactions are removed every 5 cycles and resolved on 2% agarose gels run for 30 min., and amplicons are visualized under UV light. Expression is then calculated as percent of CON, i.e., 100×(lowest number of cycles required to visualize an amplicon for CON samples/lowest number of cycles required to visualize and amplicon for XPC samples). Invasion gene expression assays were performed in triplicate for both groups (XPC and CON) in each of the 3 separate experiments, with rpoS used as the housekeeping gene control whose expression does not change significantly. That is, rpoS amplicons are typically observed at 25 to 30 cycles whereas hilA amplicons were typically observed at a wider range (10 to 35) of cycles. Data were pooled in order to calculate the Mean±SEM for three experiments performed separately.

On D21, 28, 35, 42, and 49, approximately 20% of Salmonella recovered from broiler chickens were assessed for resistance to chloramphenicol at the breakpoint concentration (32 μg/mL). Chloramphenicol was chosen since resistance to this antibiotic is encoded by the SGI1 integron present in the input Salmonella isolate. Individual black-centered colonies from XLD plates (n=96/treatment group) were inoculated into an individual well of a 96-well dish containing 200 μL of LB broth. Bacteria were grown statically overnight at 37° C. to an OD600 equal to approximately 0.3, which corresponds to a concentration of 3×108 CFU/mL. Approximately 3 μL of the growth was pin-replicated into a fresh 96-well dish in which each well contained 32 m/mL of chloramphenicol in 200 μL of LB broth. Percent chloramphenicol resistance was calculated as 100×(number of wells in which Salmonella grew in the presence of chloramphenicol/96). Chloramphenicol susceptibility assays were performed for both groups (XPC and CON).

To determine if the changes in chloramphenicol resistance were due to loss of the SGI1 (Salmonella genomic island 1) integron from the input strain, a PCR assay was performed to determine the qualitative presence of the SGI1 integron. Recovered Salmonella colonies were individually inoculated into LB broth in 96-well dishes in the absence of chloramphenicol. Bacterial growth was then subjected to a qualitative PCR assay developed and previously described by Carlson et al. 1999 (Mol. Cell. Probes. 1999; 13:213-222). Percent SGI1(+) was calculated as 100×(number of wells in which Salmonella yielded an SGI1-specific amplicon visualized using agarose gel electrophoresis/96). SGI1 prevalence was determined for both groups (XPC and CON) in each of the 3 separate experiments, with the input strain used as a positive control.

For data in which assessments were performed on multiple d (antibiotic resistance, invasion, and fecal shedding), statistical comparisons were made using a repeated measures analysis of variance with Tukey's ad hoc test for multiple comparisons. For data involving single measurements from each group (large intestinal carriage), statistical comparisons were performed using a student's t test (GraphPad). Body weight data were analyzed by the general linear model procedure of SAS software (Version 8.02, SAS Institute, Cary, N.C.) with Treatment (CON or XPC), Trial, and the interaction Treatment×Trial considered as the main effects. Absolute weight data were transformed to common logarithms prior to analysis. Mean separations were accomplished using LSMEANS with Tukey's correction. For all variables under analysis, significant differences were defined at P≤0.05. Statistical trends were consistent when the 3 sets of experiments were examined independently (data not shown).

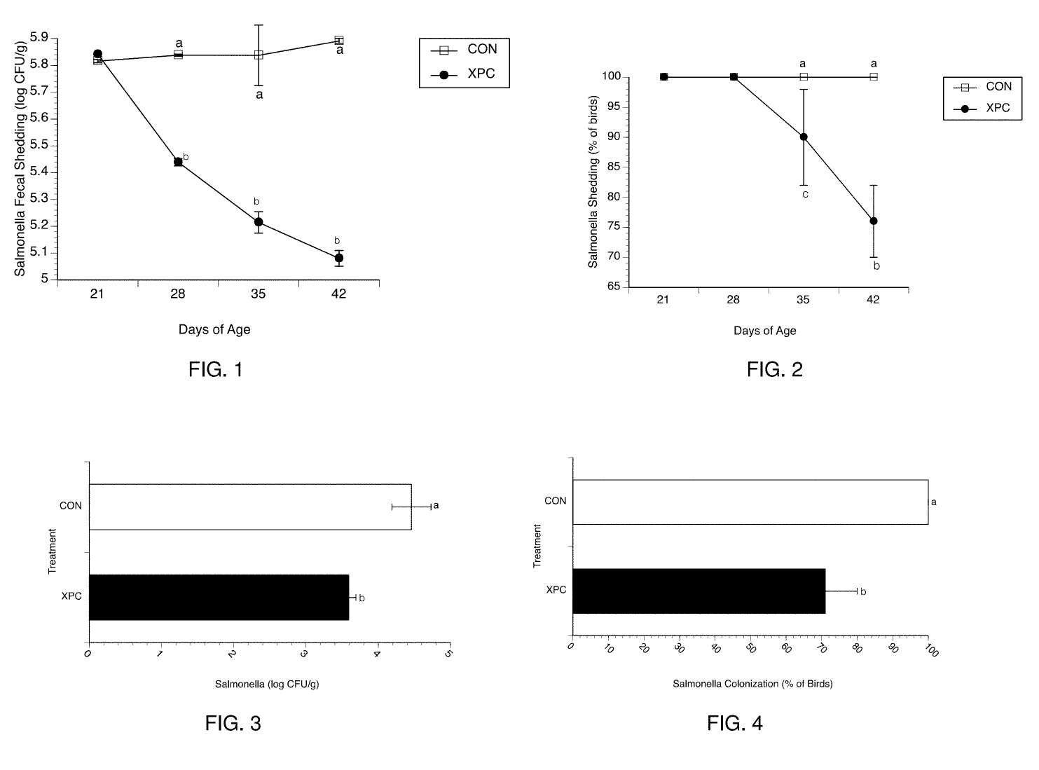

Fecal shedding of Salmonella is a potential source of this pathogen for humans who consume poultry products. On D2, 9, and 16, birds were orally inoculated with Salmonella Typhimurium and on D21 they were assigned to either the CON or XPC dietary treatment. As expected, no significant differences were observed for fecal shedding of Salmonella between chicks assigned to CON and XPC on D21 (6.5×105 versus 6.9×105 CFU/g, respectively), as dietary treatments did not start until D21 (FIG. 1). FIG. 1 demonstrates Salmonella fecal shedding (CFU/g) in broilers fed with and without Diamond V Original XPC at an inclusion rate of 1.25 kg/MT. Birds were orally inoculated with multiple antibiotic-resistant Salmonella Typhimurium on D2, 9, and 16. Dietary treatments, CON (n=57) or XPC (n=57), were applied on Day 21 and Salmonella were isolated from feces (using XLD agar) on D21, 28, 35, and 42. Data represent the Mean±SEM for three experiments performed separately wherein a,bP<0.01. Significant differences (P<0.05) in fecal shedding were observed between CON and XPC on D28, 35, and 42 with lower fecal shedding of Salmonella in birds fed XPC when compared to the CON-fed birds (FIG. 1). On D28, the XPC-fed group had a 2.4-fold decrease (0.38 on a log 10 scale) in shedding compared to CON-fed birds (2.7×105 versus 6.9×105 CFU/g, respectively). The greatest difference was observed on D42, with an approximately 7.5-fold decrease (0.88 on a log 10 scale) in shedding for chicks fed XPC (7.7×105 versus 1.2×105 CFU/g, respectively). The relative prevalence of fecal shedding on D49 (as indirectly measured by large intestinal carriage) was significantly less (P<0.05) in birds fed XPC than CON (76% vs. 100%, respectively) (FIG. 2). FIG. 2 demonstrates prevalence of Salmonella fecal shedding in broilers fed with and without Diamond V Original XPC at an inclusion rate of 1.25 kg/MT. Birds were orally inoculated with multiple antibiotic-resistant Salmonella Typhimurium on D2, 9, and 16. Dietary treatments, CON (n=57) or XPC (n=57), were applied on D21 and Salmonella were isolated from feces (using XLD agar) on D21, 28, 35, and 42. Data represent the Mean±SEM for three experiments performed separately. a,bP<0.01, a,c0.01<P<0.05.

Since Salmonella carriage in the large intestine can be a source of contamination for poultry meat, the Salmonella load in each bird was determined at the end of the study. On D49, large intestinal sections were excised and subjected to Salmonella culture and enumeration that quantifies the intestinal load of Salmonella. As shown in FIG. 3, large intestinal carriage was significantly less (P<0.05) in birds fed XPC compared to CON (3,875 vs. 29,023 CFU/g of intestine, respectively; 0.88 log 10 reduction). FIG. 3 demonstrates large intestinal colonization by Salmonella on Day 49 in broilers fed with and without Diamond V Original XPC at an inclusion rate of 1.25 kg/MT. Dietary treatments, CON (n=57) or XPC (n=57), were applied on D21. On D49, all birds were euthanized and a section of the large intestine was removed and selectively and enumeratively cultured for Salmonella using XLD agar. Data represent the Mean±SEM for three experiments performed separately. a,bP<0.01. The relative prevalence of large intestinal carriage was significantly less (P<0.05) in birds fed XPC versus CON (71% vs. 100%, respectively) (FIG. 4). FIG. 4 demonstrates prevalence of large intestinal colonization by Salmonella on Day 49 in broilers fed with and without Diamond V Original XPC at an inclusion rate of 1.25 kg/MT. Dietary treatments, CON (n=57) or XPC (n=57), were applied on D21. On D49, all birds were euthanized and a section of the large intestine was removed and selectively and enumeratively cultured for Salmonella using XLD agar. Data represent the Mean±SEM for three experiments performed separately. a,bP<0.01.

To determine if the treatment had an effect on broiler performance as previously reported after challenge with a different Salmonella serovar), broilers in each group were individually weighed on D21 and 49 and these data are presented in Table 1. No significant 2-way interaction between Treatment and Trial were observed for any of the measurements, therefore only Treatment effects are presented in Table 1. Because of the serpentine assignment format, no significant differences in body weight (BW) were observed between birds assigned to XPC and CON groups on D21. By D49, XPC-fed birds were significantly heavier than CON-fed birds (3.504 vs. 3.243 kg, respectively). Birds fed XPC from D21 to 49 exhibited significantly heavier weight gain than CON-fed birds (2.613 vs. 2.343 kg, respectively).

TABLE 1
Body weights (kg), body weight gains (kg), and
statistical probabilities for broilers fed with
and without Diamond V Original XPC ™ and
challenged with Salmonella Typhimurium.

| | Treatment | | |
|---|---|---|---|
| Variable^{1,2} | CON³ | XPC^{3,4,5} | P-value |
| BW 21d | 0.896 ± 0.018 | 0.881 ± 0.017 | 0.5077 |
| BW 49d | 3.243 ± 0.045^{b} | 3.504 ± 0.044^{a} | <0.0001 |
| BW Gain (21-49d) | 2.343 ± 0.045^{b} | 2.613 ± 0.043^{a} | 0.0122 |

¹Means ± SEM.
²Means across rows within the same variable column with no common superscript differ significantly (P < 0.05).
³Sample size: CON (n = 57) and XPC (n = 57)
⁴Diamond V Original XPC inclusion rate was 1.25 kg/MT for all diets in the XPC treatment group.
⁵Dietary treatment was applied on Day 21.

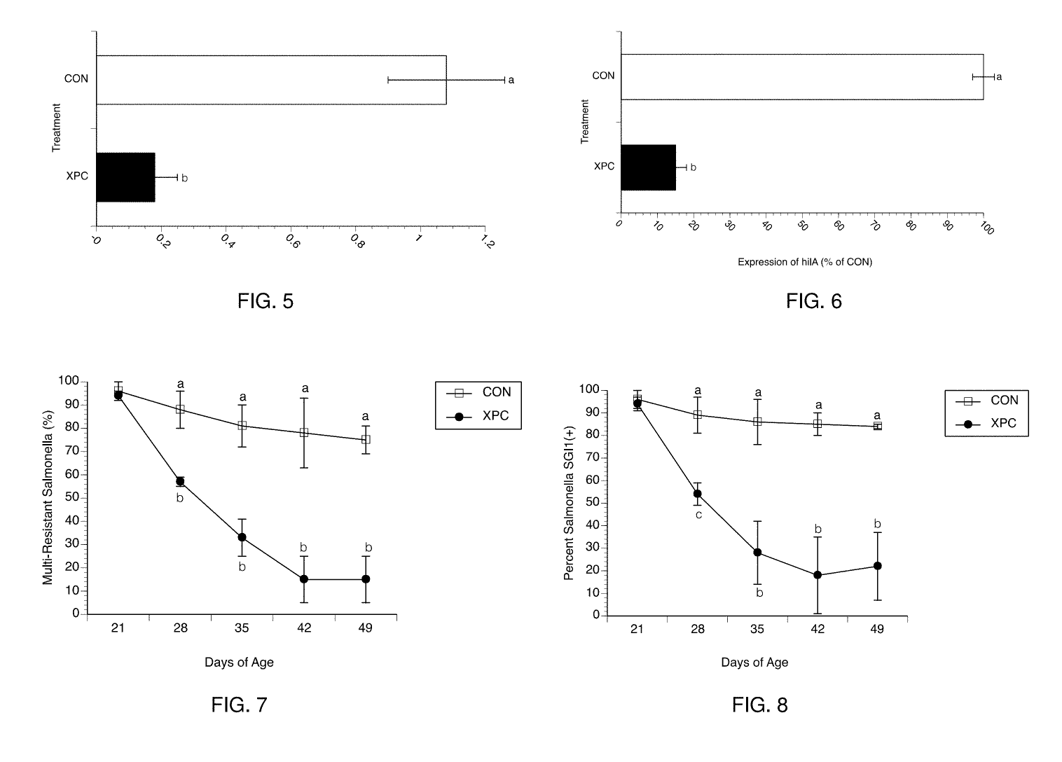

At all 5 time points in which Salmonella were recovered and quantitated from the birds (D21, 28, 35, 42 and 49), presumptive colonies were collected by group and then subjected to a tissue culture invasion assay. No significant differences were observed for invasiveness of Salmonella on D21 (1.06 vs. 1.03%), as dietary treatments did not start until D21. Significant differences (P<0.05) in invasiveness were observed between CON and XPC (1.08% vs. 0.18%, respectively; i.e., a 0.78 reduction on a log₁₀ scale) on D49 with Salmonella exhibiting decreased invasiveness following isolation from birds fed XPC when compared to the CON-fed birds (FIG. 5). FIG. 5 demonstrates tissue culture invasiveness of Salmonella recovered from broilers challenged with Salmonella Typhimurium and fed with and without Diamond V Original XPC at an inclusion rate of 1.25 kg/MT. Dietary treatments, CON (n=57) or XPC (n=57), were applied on D21 and continued until D49 when intestinal samples were taken and approximately 30% of the Salmonella recovered (approximately 10⁵ CFU) were subjected to the invasion assay using mammalian tissue culture cells and a multiplicity of infection equal to at least one. Percent invasion is a calculated as 100×(number of Salmonella recovered from within the tissue culture wells/number of Salmonella added to the tissue culture wells). Data represent the Mean±SEM for three experiments performed separately. ^{a,b}P<0.01.

This decrease in invasiveness of Salmonella coincided with a decrease in the expression of hilA (FIG. 6), a major regulator of Salmonella virulence for mammalian hosts. FIG. 6 demonstrates expression of hilA in Salmonella recovered from feces of broilers challenged with Salmonella Typhimurium and fed with and without Diamond V Original XPC at an inclusion rate of 1.25 kg/MT. Dietary treatments, CON (n=57) or XPC (n=57), were applied on D21 and continued until D49 when intestinal samples were taken and approximately 20% of the Salmonella recovered were subjected to the RNA isolation and semi-quantitative RT-PCR as previously described (Carlson et al., 2007). Expression was then calculated as percent of CON, i.e., 100×(number of cycles required to visualize an amplicon for CON samples/number of cycles required to visualize an amplicon for XPC samples). Data represent the Mean±SEM for three experiments performed separately. a,bP<0.01.

Salmonella recovered from birds were subjected to a chloramphenicol susceptibility assay. This line of study was pursued since the input strain bears a chloramphenicol resistance-encoding genetic structure (SGI1) that can be dislodged from Salmonella based on previous studies. FIG. 7 reveals a similar prevalence of resistant Salmonella on D21 in both groups (96 vs. 94%) followed by a decrease in the prevalence of chloramphenicol resistance (P<0.05) in Salmonella recovered from broilers fed XPC on D28, 35, 42 and 49, as compared to Salmonella recovered from CON fed birds (57 vs. 88%, 33 vs. 81%, 15 vs. 78%, and 15 vs. 75%, respectively). FIG. 7 demonstrates Chloramphenicol resistance of Salmonella recovered from the feces (D21, 28, 35, and 42) or intestines (D49) of broilers challenged with Salmonella Typhimurium and fed with and without Diamond V Original XPC at an inclusion rate of 1.25 kg/MT. Dietary treatments, CON (n=57) or XPC (n=57), were applied on D21. On D21, 28, 35, 42, and 49, approximately 20% Salmonella recovered from broiler chickens were assessed for resistance to chloramphenicol at the breakpoint concentration (32 μg/mL; CLSI, 2008). Data represent the Mean±SEM for three experiments performed separately. a,bP<0.01. This reduction in chloramphenicol resistance was likely due to egress of the SGI1 integron, as presented in FIG. 8, in which about 80% of the isolates from the CON-fed birds retained SGI1 yet only 10 to 20% of isolates retained SGI1 in birds fed XPC. FIG. 8 demonstrates presence of SGI1 in Salmonella recovered from the feces (D21, 28, 35, and 42) or intestines (D49) of broilers challenged with Salmonella Typhimurium and fed with and without Diamond V Original XPC at an inclusion rate of 1.25 kg/MT. Dietary treatments, CON (n=57) or XPC (n=57), were applied on D21. Recovered Salmonella colonies were individually inoculated into LB broth in 96-well dishes in the absence of chloramphenicol. Bacterial growth was then subjected to a PCR assay developed and previously described by Carlson et al. (1999). Percent SGI1(+) was calculated as 100×(number of wells in which Salmonella yielded an SGI1-specific amplicon/96). Data represent the Mean±SEM for three experiments performed separately. a,bP<0.01, a,c0.01<P<0.05.

In summary, feeding XPC reduced the virulence and antibiotic resistance of the input Salmonella strain. Further, because feeding XPC reduced expression of the hilA gene in Salmonella, the reduced virulence of Salmonella was likely the result of reduced expression of the hilA gene. Additionally, broilers were significantly less likely to harbor large intestine Salmonella in birds fed XPC. Ultimately, these varying yet beneficial effects will have a marked positive effect on food safety in the poultry industry and future mechanistic studies will uncover the molecular bases for these effects.

### Example 2

### Effects of Feeding NaturSafe, a Saccharomyces cerevisiae Fermentation Product, on Antibiotic Resistance and Fecal Shedding of Salmonella and E. coli O157:H7, and Virulence of Salmonella in Beef Cattle

A feedlot study using heifers was conducted to examine the effects of feeding NaturSafe on antibiotic resistance and fecal shedding of Salmonella and E. coli O157:H7, and virulence of naturally occurring Salmonella. Heifers (n=1,495; 300-400 kg) were obtained from two sale barns (n=438) and one backgrounding facility (n=1,057). Cattle were shipped to a commercial feedlot and were provided water and hay ad libitum. On day 1 post-arrival, heifers were individually weighed, identified, implanted, and vaccinated using standard procedures at the feedlot. Heifers were then randomly assigned into pens in groups of five until each pen reached its optimal capacity (˜75 animals) based on bunk space and the area of the pen (14.4 inches of bunk space and 231 square feet of pen space per animal).

Two adjacent pens were designated as a single block and 10 blocks were created within the feedlot. Pens of heifers in each block were provided either a diet that contained a combination of standard industry technologies (PC), including monensin (Rumensin, Elanco Animal Health, 300 mg/head/day), tylosin (Tylovet, Huvepharma, 90 mg/head/day) and a direct-fed microbial (Bovamine Defend, Nutrition Physiology Company, 50 mg/head/day); or a diet containing 18 gm/head/day of a S. cerevisiae fermentation prototype (PRT; NaturSafe, Diamond V) without monensin, tylosin, or the direct-fed microbial. Heifers received three step-up diets prior to their final finishing diet (Table 1). All treatment feed additives were stored under manufacture recommend conditions and added to the final ration using a microingredient weight machine (Micro Beef Technologies, Amarillo, Tex.).

TABLE 1
Composition of Diets

| Ingredient, % DM | Starter | Ration 2 | Ration 3 | Finisher |
|---|---|---|---|---|
| Steam-flaked corn | 30.2 | 45.8 | 58.6 | 66.1 |
| Wet distiller's grain | 22.2 | 19.5 | 18.0 | 18.0 |
| Alfalfa hay | 38.0 | 25.0 | 13.0 | — |
| Corn stalks | — | — | — | 4.0 |
| Corn silage | 7.0 | 7.0 | 5.0 | 4.0 |
| Tallow | — | — | 1.5 | 2.9 |
| Supplement^{1,2} | 2.6 | 2.7 | 3.9 | 5.0 |

¹Control rations were formulated to provide 300 mg of monensin (Elanco Animal Health, Greenfield, IN), 90 mg of tylosin (Zoetis Animal Health, Florham, NJ), 0.5 mg of melengestrol acetate (Zoetis Animal Health), and 50 gm Bovamine Defend (Nutrition Physiology Company, Overland Park, KS) per heifer daily throughout the study, and 250 mg of ractopamine hydrochloride (Zoetis Animal Health) per heifer daily during the last 28 days on feed.
²Rations containing PRT were formulated to provide 18 gm of a Saccharomyces cerevisiae fermentation prototype (Diamond V, Cedar Rapids, IA) and 0.5 mg of melengestrol acetate (Zoetis Animal Health) per heifer daily throughout the study, and 250 mg of ractopamine hydrochloride (Zoetis Animal Health) per heifer daily during the last 28 days on feed.

During the study, heifers were monitored for illness and treated as per recommendations by a veterinarian. Heifers that responded to treatment were returned to the study while non-responders were removed from the study. Morbidities and mortalities were indistinct between the two groups (data not shown).

At the conclusion of the study, heifers were shipped 145 miles to a commercial abattoir on two separate dates that were three weeks apart. These shipping dates corresponded to 125 and 146 days on study for the first and second groups, respectively. An equal number of pens per treatment group were shipped on each date (n=5 per treatment). Shipments and carcass processing occurred on a pen-by-pen basis.

Fecal swabs were collected on the rail from 20 animals per pen (replicate). Samples were collected from every third or fourth animal within a replicate. Fecal samples were collected using a 3M-sponge stick pre-saturated with buffered peptone water. Sponge sticks were inserted into the rectum (recto-anal junction) to collect the sample. After the sample was collected, the sponge was placed into a pre-labeled bag containing buffered peptone water. The bag was closed and placed into a cooler.

Subiliac lymph nodes and the surrounding tissue were collected post evisceration. Sample collection began with the first carcass in each replicate and continued with every third or fourth carcass within that replicate. Lymph nodes were placed into pre-labeled Whirlpak bags. The bags were closed and placed in a cooler. Fecal swabs and lymph node samples were then immediately shipped on ice for microbiological analyses.

Salmonella spp. were enumerated from every fecal swab sample and lymph node collected (20 per pen; 200 per treatment) using selective agar (XLD) methods described in Example 1. Approximately 0.3 gm of feces or lymph node were collected on a sterile cotton swab and then aseptically transferred into 10 mL Lennox broth and an aliquot of the broth was immediately plated on XLD agar, incubated overnight at 37° C., and subjected to enumeration by manual counting of black-centered colonies the next day. Load was then determined as (colonies recovered)×(the dilution factor)/gm of feces or lymph node. Prevalence was calculated as percent of heifers harboring any Salmonella and was compiled across pens within a treatment group.

E. coli O157:H7 was enumerated in 100 of the swab samples (five per pen; 50 swab samples per treatment) using selective media (Sorbitol-MacConkey agar) and a PCR targeting E. coli O157:H7 virulence genes. Approximately 0.3 gm of feces were transferred into enrichment broth and an aliquot of the broth was plated on sorbitol-MacConkey agar, incubated overnight at 37° C., and subjected to enumeration by manual counting of non-fermenting colonies the next day. From each pen-specific set of agar plates, 96 colonies were selected and subjected to the PCR targeting E. coli O157:H7 virulence genes. Load was then determined as (colonies recovered×the dilution factor×the percent of colonies yielding an E. coli O157H7-specific amplicon within the pen)/gm of feces. Prevalence was calculated as percent of heifers harboring any E. coli O157:H7 within a pen, and was compiled across pens within a treatment group.

Approximately 50% of the recovered Salmonella were subjected to a standard antibiotic protection-based tissue culture invasion assay adapted for use with both antibiotic-susceptible and antibiotic-resistant Salmonella as detailed in Example 1. Colonies were collected en masse, on a pen-by-pen basis, into nutrient broth and then immediately incubated for 1 hour with HEp-2 tissue culture cells at 37° C. Bacteria were recovered from inside tissue culture cells via cell lysis, incubated on XLD agar overnight at 37° C., and enumerated the next day. Percent invasion was determined as (number of black-centered colonies recovered from inside cells/number of colonies added to cells)×100.

In order to correlate the virulence of Salmonella recovered from cattle with gene expression events in the pathogen, approximately 10% of the recovered Salmonella isolates were subjected to an assay that quantitates the expression of hilA (a key regulator of Salmonella invasion genes). RNA was extracted from the isolates that were collected en masse on a pen-by-pen basis, and then subjected to a semi-quantitative RT-PCR targeting the hilA transcript as described in Example 1.

Approximately 20% of the recovered Salmonella were individually subjected to micro-broth assays with individual antibiotics (Florfenicol, Ceftiofur, and Enrofloxacin) at breakpoint concentrations. Colonies that grew in the breakpoint concentrations were deemed to be resistant. Percent resistant were then determined as (number of resistant colonies/number of colonies examined)×100. Data were compared across pens and between groups.

Nearly 20% of the recovered Salmonella were individually subjected to PCR assays that detect the presence of genes related to Dublin [Akiba et al. J. Microbiol. Methods 2011 April; 85(1):9-15], Typhimurium [Akiba et al. J. Microbiol. Methods 2011 April; 85(1):9-15], and Newport [PLoS One. 2013; 8(2):e55687] serotypes. Colonies yielding a specific PCR amplicon(s) were deemed to belong to the ascribed serotype. Percent belonging to the serotype were then determined as (number of colonies yielding a specific amplicon/number of colonies examined)×100. Data were compared across pens and between groups.

Statistical comparisons were made using an analysis of variance with Tukey's ad hoc test for multiple comparisons. Significant differences were defined at P≤0.05.

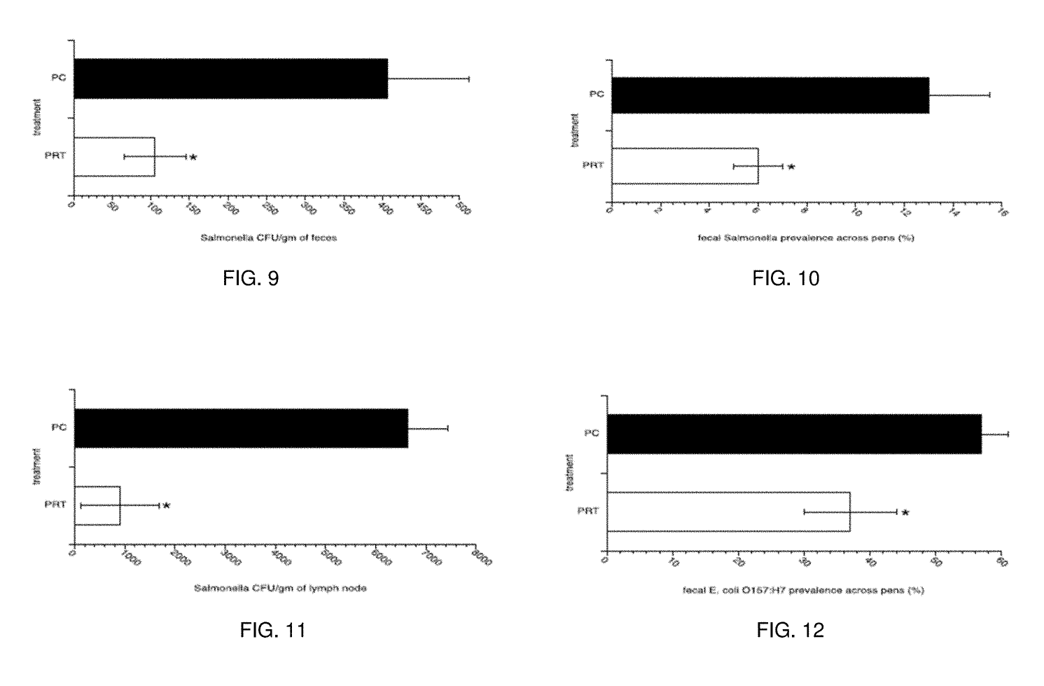

In this study, fecal shedding of Salmonella was evaluated in 200 heifer's postmortem from each treatment group. As shown in FIG. 9, fecal shedding of Salmonella was significantly less (P<0.05) in cattle fed PRT (105 versus 405 CFU/gm of feces, respectively). The relative prevalence of fecal shedding was significantly less (P<0.05) in heifers fed PRT (6 versus 13%, respectively) as per FIG. 10.

Since Salmonella lymph node carriage can be a source of contamination of ground beef, Salmonella load was determined in the subiliac lymph nodes of 200 carcasses from each treatment group. As shown in FIG. 11, lymph node infiltration was significantly less (P<0.05) in carcasses from heifers fed PRT (902 versus 6,642 CFU/gm of lymph node, respectively). The percent of Salmonella-bearing lymph nodes was significantly less (P<0.05) in carcasses from heifers fed PRT (4 versus 14%, respectively) as per FIG. 12.

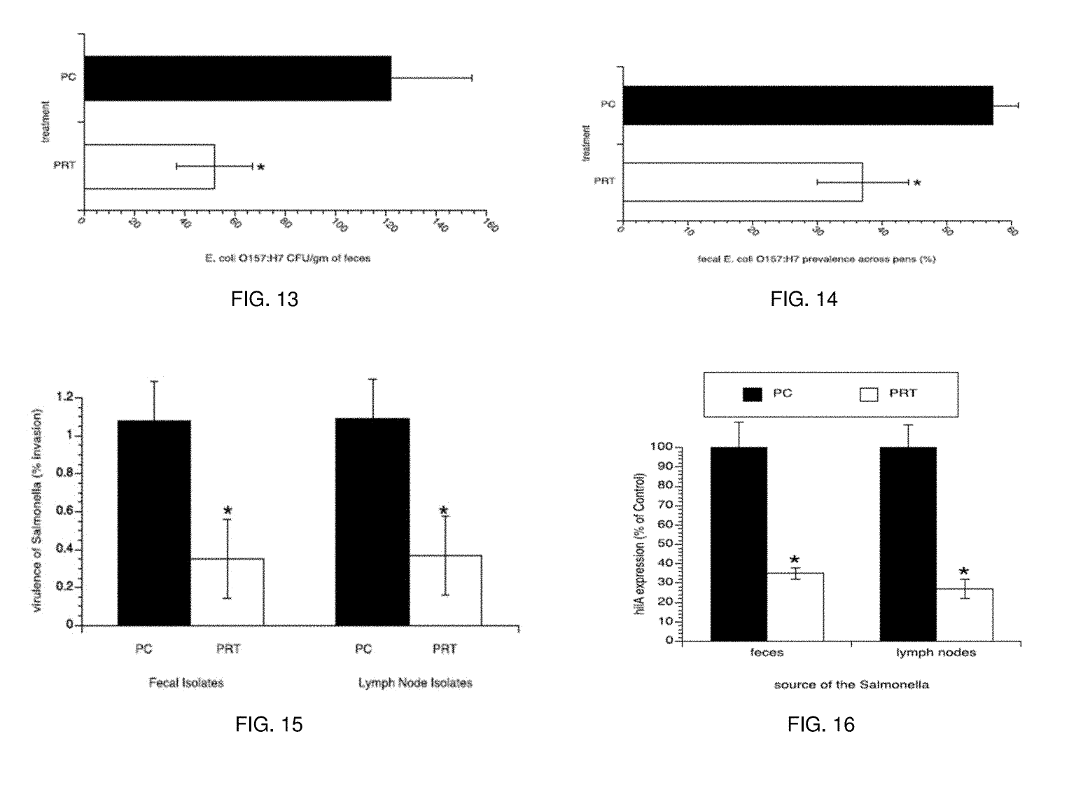

To determine if PRT had an effect on the presence of E. coli O157:H7 in the feces of the heifers, fecal samples (100 per treatment group) were quantitatively examined for the presence of this critical foodborne pathogen. As shown in FIG. 13, heifers fed PRT had a statistically lower (P<0.05) E. coli O157:H7 fecal load than heifers fed PC (52 versus 122 CFU/gm of feces, respectively). FIG. 14 reveals a decreased prevalence (P<0.05) of E. coli O157:H7 in heifers fed PRT when compared to those receiving the PC diet (37 versus 57%, respectively).

In order to compare the virulence of Salmonella recovered from cattle, the isolates were subjected to an assay that predicts the ability of Salmonella to invade gut epithelial cells, which is a hallmark of Salmonella virulence. The effects of PRT on virulence were examined due to the ability of SCFP to increase butyrate in the intestine. Research has shown that butyrate can decrease the Salmonella invasion gene (hilA) expression in vitro, which results in the decreased ability of Salmonella to invade cells. In the current study, invasiveness of Salmonella was significantly less (P<0.05) in Salmonella recovered from the feces and lymph nodes of cattle fed PRT (FIG. 15). This decrease in invasiveness coincided with a decrease in the expression of hilA (FIG. 16), a major regulator of Salmonella virulence for mammalian hosts.

To assess the possibility that PRT inhibits antibiotic resistant Salmonella or induces the expulsion of antibiotic resistance elements from Salmonella, isolates recovered from cattle were subjected to an antibiotic susceptibility assay that utilized three individual antibiotics (Ceftiofur, Enrofloxacin, and Florfenicol). These three antibiotics were chosen given their extended spectra and importance in bovine therapeutics. Additionally, two of the three antibiotics tested (Ceftiofur and Enrofloxacin) have counterparts important for human therapeutics (Ceftriaxone and Ciprofloxacin, respectively).

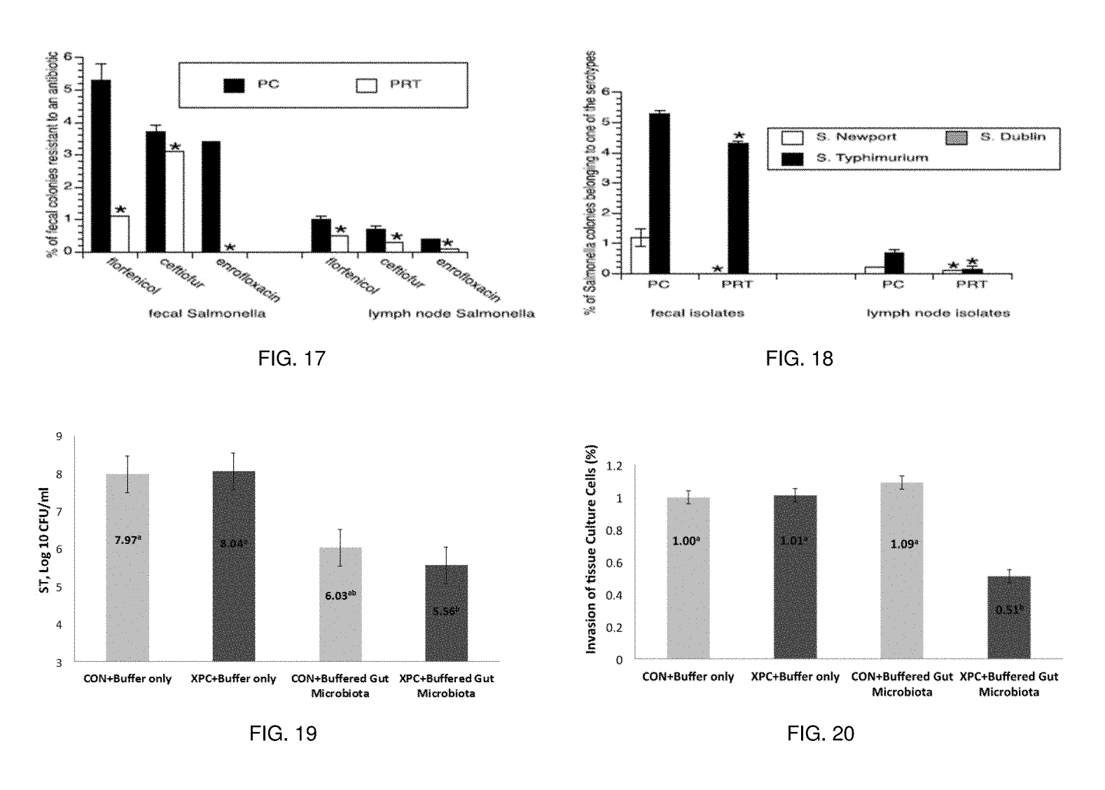

FIG. 17 reveals a decrease in the prevalence of resistant (P<0.05) Salmonella recovered from heifers fed PRT for all three antibiotics. This figure represents isolates from both feces and lymph nodes. It is of note that resistance of these antibiotics was, in general, more prevalent in the fecal isolates when compared to the lymph node isolates.

Salmonella isolates recovered from the feces or lymph nodes of cattle were subjected to PCR assays targeting three serotypes (Dublin, Newport, and Typhimurium). As shown in FIG. 18, the prevalence of two of these serovars was diminished in heifers fed PRT, regardless of the source of the isolates. No S. Dublin were isolated from feces and only one colony of S. Dublin was isolated from lymph nodes. Thus, statistical evaluations are not presented for this minor subsection of the study.

Salmonella and E. coli O157:H7 are insidious problems for the beef industry and represent critical food safety hazards. Salmonella and E. coli O157:H7 can be shed in fecal material that can contaminate the carcass during processing. Salmonella is also harbored in the lymph nodes, which can lead to contamination of ground beef. Therefore, identifying mitigation strategies for both pathogens is needed especially considering the covert nature of Salmonella lymph node infiltration.

In this study, the anti-Salmonella and anti-E. coli O157:H7 effects of NaturSafe (PRT) were examined and two critical indicators of Salmonella contamination (fecal shedding and lymph node infiltration) were significantly reduced by NaturSafe. In this study, heifers fed PC shed a higher number of Salmonella and E. coli O157:H7 and had more Salmonella present in the lymph nodes, which ultimately increases the risk of pathogen transmission to humans that ingest beef.

Other significant and unique findings in this study were the reduction in virulence and antibiotic resistance in Salmonella recovered from heifers fed PRT. Reduced virulence was detected by diminished tissue culture invasion with a concomitant reduction in the expression of hilA. The observed magnitude of decreased invasiveness is likely to increase the infectious dose of Salmonella for a human as evidenced by our prior study, in which this level of diminished invasiveness altered the murine LD₅₀ approximately 5-fold.

In summary, NaturSafe reduced the virulence and antibiotic resistance of recovered Salmonella. Further, because feeding NaturSafe reduced expression of the hilA gene in Salmonella, the reduced virulence of Salmonella was likely the result of reduced expression of the hilA gene. NaturSafe fed feedlot heifers were significantly less likely to shed Salmonella and harbor this pathogen in the lymph nodes. The anti-shedding effect of NaturSafe was also observed for E. coli O157:H7. Ultimately, these beneficial effects will have a marked positive effect on food safety in the beef industry.

### Example 3

### Gut Microbiota-Mediated Suppression of Virulence and Antibiotic Resistance of Salmonella Typhimurium DT104 by Original XPC in an In Vitro Poultry Model

An in vitro model was utilized model to determine whether the effects of XPC on virulence and antibiotic resistance of Salmonella Typhimurium (ST) are: (1) independent of the host animal; and, (2) dependent on the presence of gut microbiota. Multiple antibiotic resistant (MAR) ST strain DT104 was used as the challenge organism. Freshly voided excreta from 10 broilers (35 days of age) was combined and served as the source of gut microbiota. Under anaerobic conditions, buffered media (pH 6.8) with excreta or buffered media alone (30 mL); a pre-digested (pepsin, pH 2.0; pancreatin, pH 7.0) broiler finisher diet (0.15 g) with (XPC) or without XPC (CON; n=5); and MAR ST strain DT104 (1×104 CFU/ml, final concentration) were added to vessels and incubated with continuous mixing (39° C.; 24 h).

For re-isolation and enumeration of Salmonella, serial dilutions of the tube contents were plated onto modified XLT4 agar (XLT4 agar base Difco#0234-17 and XLT4 supplement Difco#0353-72), containing 30 mg/L novobiocin and the plates incubated at 37° C. for 48 h. Re-isolated Salmonella was subjected to (1) antibiotic resistance testing, using chloramphenicol at the breakpoint concentration of 32 μg/mL (CLSI, 2008), and (2) PCR quantitation of the presence of the SGI1 integron that confers resistance to the input strain as detailed in Example 1. Re-isolated Salmonella was also subjected to (1) a human epithelial type 2-cell invasion assay, which predicts ability of Salmonella to cause disease in a human, and (2), quantification of the expression of the hilA gene for treatments from buffered fecal incubation as detailed in Example 1.

Expression of hilA was calculated as 100×(number of cycles required to visualize an amplicon for CON samples/number of cycles required to visualize and amplicon for CON or XPC).

The study was performed 3 times, the combined data analyzed by ANOVA in JMP (SAS Institute, Cary, N.C.) and differences between means determined using Tukey HSD test. Differences in hilA expression were determined using the Student's T test. The entire trial was performed 3 times, and the combined data analyzed using JMP (SAS).

When treatments were incubated in buffer only, ST from CON and XPC had similar (P>0.05) invasiveness (1.0%); however, antibiotic resistance was marginally lower (P<0.05) for XPC (84%) than CON (98%). In the presence of gut microbiota, XPC reduced invasiveness of ST (P<0.05) from 1.1% (CON) to 0.5% (XPC) and antibiotic resistance (P<0.05) from 90% (CON) to 35% (XPC).

ST grew approximately 1.5 to 2.0 logs in the presence of gut microbiota, but grew by up to 4 logs in buffered media without gut microbiota (FIG. 1). XPC tended to reduce ST growth in the presence of gut microbiota but had no effect at all in buffered media only (FIG. 19).

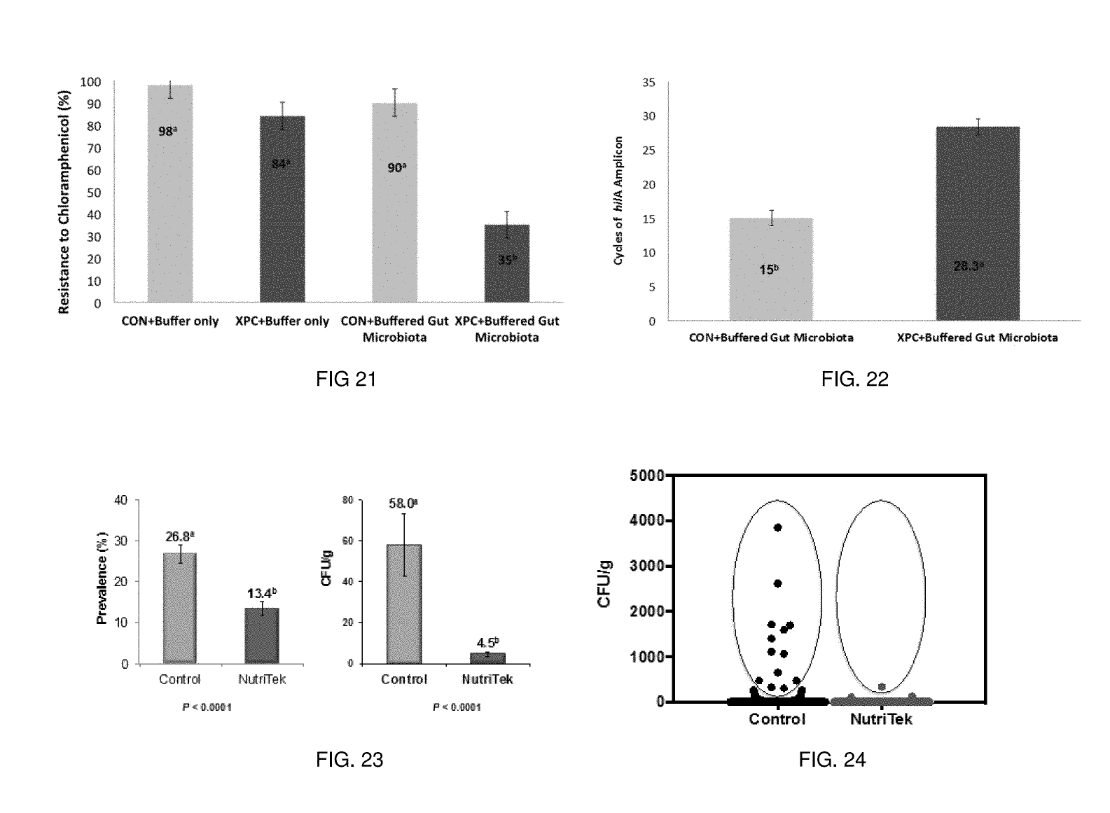

ST colonies recovered from CON and XPC incubated in buffered media only had no differences (P>0.05) in virulence (FIG. 20) and antibiotic sensitivity (FIG. 21). In the presence of gut microbiota, XPC reduced invasiveness of ST (P<0.05) from 1.1% (CON) to 0.5% (XPC; FIG. 20), and antibiotic resistance (P<0.05) from 90% (CON) to 35% (XPC; FIG. 21). Inhibition of virulence appeared to have been due to the decreased expression of hilA. Restoration of antibiotic sensitivity appears to be due to an “expulsion” of the SGI1 integron (FIG. 22).

In summary, Original XPC suppressed virulence and re-established antibiotic sensitivity of Salmonella Typhimurium DT104, via its influence on gut microbiota, outside the animal. There was no evidence of a direct effect of Original XPC on Salmonella. Further, because XPC reduced expression of the hilA gene in Salmonella, the reduced virulence of Salmonella was likely the result of reduced expression of the hilA gene.

### Example 4

### Effects of Feeding a NutriTek, a Saccharomyces cerevisiae Fermentation Product, to Dairy Cows on Numbers, Prevalence, Virulence and Antibiotic Resistance of Salmonella and E. coli in 5 Commercial Dairy Herds

A pathogen survey was conducted on 5 commercial dairy farms in order to establish the effects of feeding NutriTek on pathogen load, virulence and antibiotic resistance in a commercial setting. The pathogens surveyed for were Salmonella and E. coli. Multiparous cows were split in to a control and NutriTek group and baseline pathogens levels enumerated from fecal swabs collected from cows during December 2016 (Data not shown). Cows were then fed the same diet with (NutriTek; 19 g/h/d)) or without (Control) NutriTek at the recommended daily rate of 19 g per head per day, for 60 days. Following the 60 days, fecal swabs were again collected from cows on all farms using the techniques described in Example 2. Fecal swabs were handled as detailed in Example 2. Salmonella and E. coli enumeration (prevalence and numbers) was concluded as detailed in Example 2. Colonies recovered from the fecal swabs were subjected to antibiotic resistance testing for both E. coli and Salmonella, and to virulence testing, for Salmonella only. Salmonella virulence testing was concluded as detailed in Example 1. Because Ceftiofur, Florfenicol and Enrofloxacin are antibiotics of particular significance to human health, E. coli and Salmonella colonies obtained from the fecal swabs were tested for resistance to these compounds using standard methods known to those skilled in the art (CLSI 2017; Performance standards for antimicrobial disk and dilution susceptibility tests. Wayne, Pa.).

The virulence, prevalence, numbers and antibiotic resistance data collected after 60 d of feeding NutriTek was pooled across the five farms. Statistical analysis was then performed to determine the difference between Control and NutriTek treatments in the parameters of interest.

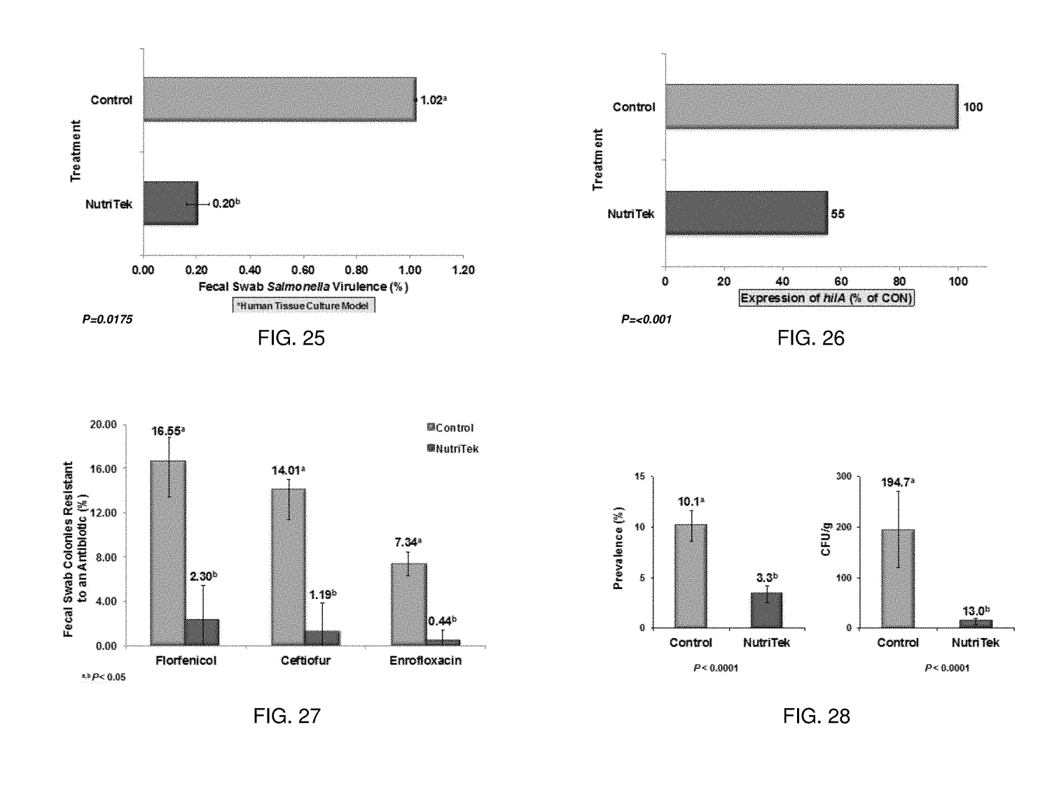

FIGS. 23-27 illustrate the treatment means of pooled results for Salmonella across the 5 commercial dairy farms. Cows fed NutriTek had a 50% reduction in prevalence, and a reduction of more than 90% in numbers, of Salmonella (FIG. 23). NutriTek reduced numbers of average numbers of Salmonella by reducing the number of samples with high concentrations of Salmonella (FIG. 24). Virulence of the Salmonella isolated from NutriTek cows was on average 20% of that measured in Salmonella isolated from cows fed the Control (FIG. 25). This marked reduction in virulence of Salmonella isolated from cows fed NutriTek was associated with a 50% reduction in expression of the hilA gene for this treatment (FIG. 26).

Significantly, Florfenicol, Ceftiofur and Enrofloxacin resistant Salmonella (FIG. 27) recovered from cows fed NutriTek were reduced to approximately 20% or less of those recovered from control diet fed cows.

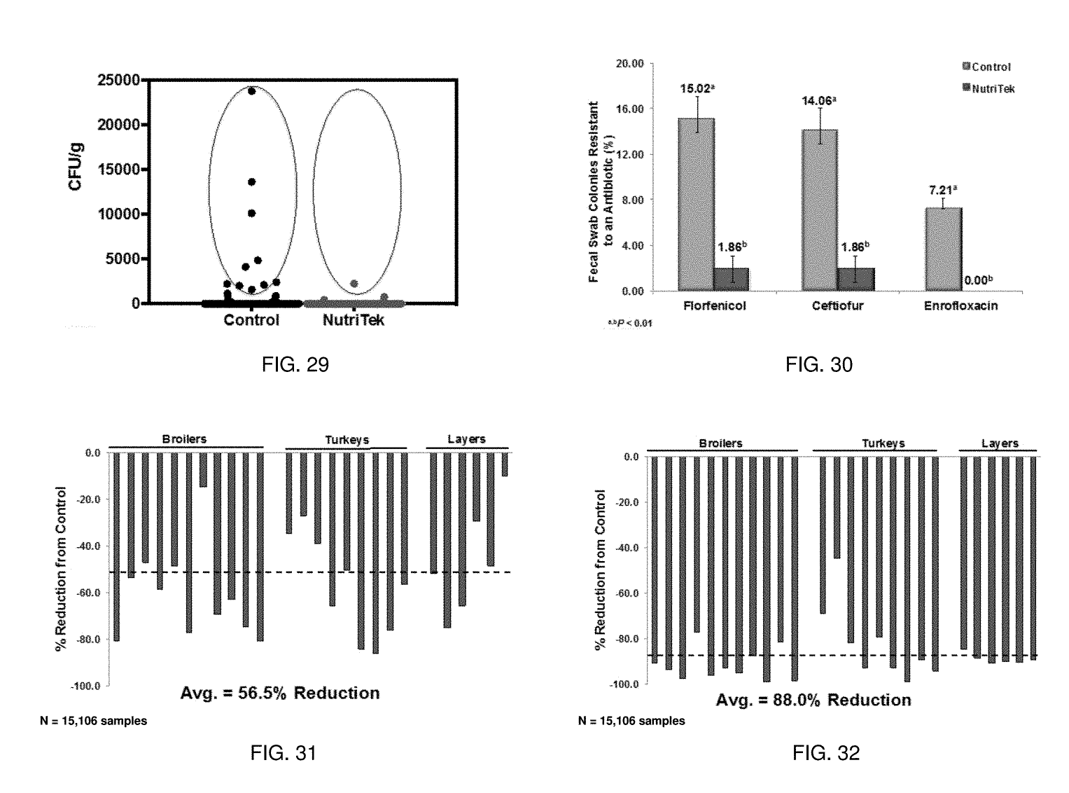

FIGS. 28-30 illustrate the treatment means of pooled results for E. coli across the 5 commercial dairy farms. Clearly, feeding NutriTek reduced prevalence and numbers of E. coli by more than 65% and more than 90%, respectively (FIG. 28; all P<0.0001). Further, a dot plot of E. coli numbers (FIG. 29) demonstrated that feeding NutriTek dramatically reduced the number of samples with high E. coli concentrations. Pooled antibiotic resistance data from all 5 trials (FIG. 30) also illustrated that the percentage of E. coli colonies resistant to all 3 antibiotics tested was substantially and significantly (P<0.05) reduced in samples obtained from cows fed NutriTek.

In summary, feeding NutriTek reduced the virulence of Salmonella, and resistance of recovered Salmonella and E. coli to Florfenicol, Ceftiofur and Enrofloxacin. Further, because feeding NutriTek reduced expression of the hilA gene in Salmonella, the reduced virulence of Salmonella was likely the result of reduced expression of the hilA gene. NutriTek-fed dairy cows were significantly less likely to shed Salmonella. The anti-shedding effect of NutriTek was also observed for E. coli. Ultimately, these beneficial effects will have a marked positive effect on food safety in dairy industry.

### Example 5

### Effects of Feeding XPC, a Saccharomyces cerevisiae Fermentation Product, on Numbers, Prevalence, Virulence and Antibiotic Resistance of Salmonella on Commercial Poultry Operations

Field trials were performed at multiple broiler, layer and turkey farm operations, using matched houses or farms, to confirm the effects of feeding XPC on numbers, prevalence, virulence and antibiotic resistance of natural occurring Salmonella in a commercial setting. A total of 24 companies participated in this testing. On each farm, birds were fed the commercial diet in use at the specific farm, with (XPC; 1.25 kg/MT) or without XPC (Control), from one day of age to market maturity. For the layer operations, the cloaca of the birds were swabbed for microbial evaluation, and in the case of the broilers and turkeys, ceca were collected at the processing plant. Additionally, for layers, environmental samples were collected within each barn using established procedures known by those skilled in the art. All samples were shipped overnight on ice where they evaluated for Salmonella numbers, prevalence, virulence and antibiotic resistance using the procedure detailed in Example 1. Because Ceftiofur, Florfenicol and Enrofloxacin are antibiotics of particular significance to human health, E. coli and Salmonella colonies obtained from the fecal swabs were tested for resistance to these compounds using standard methods known to those skilled in the art (CLSI 2017; Performance standards for antimicrobial disk and dilution susceptibility tests. Wayne, Pa.). This was a robust commercial trial involving 318 poultry barns. In total, 15,106 samples were collected and 34,262 Salmonella colonies were tested for antibiotic resistance. Data was pooled across companies and treatment differences established by statistical analysis (SAS).

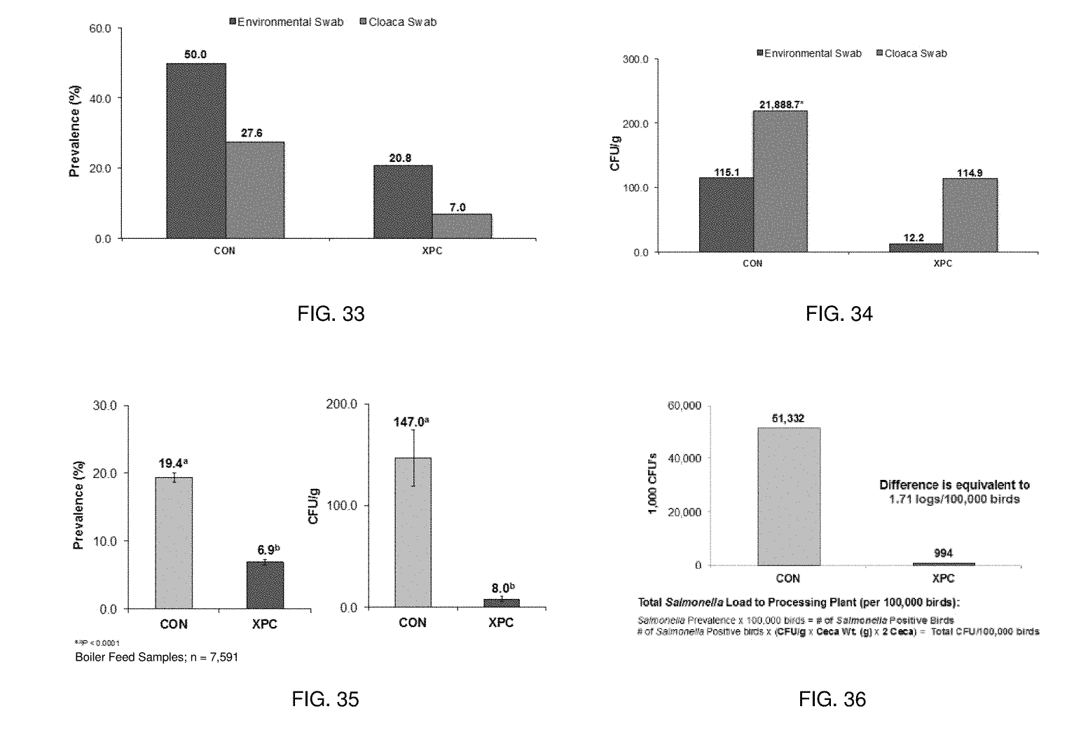

Feeding XPC reduced prevalence and numbers of Salmonella in all poultry species and all commercial operations tested (FIGS. 31 and 32). Feeding XPC reduced prevalence (FIG. 31) and numbers (FIG. 32) across all poultry species by an average of 56% and 88% respectively. Furthermore, feeding XPC to layers reduced Salmonella prevalence (FIG. 33) and numbers (FIG. 34) in both cloacal and environmental samples.

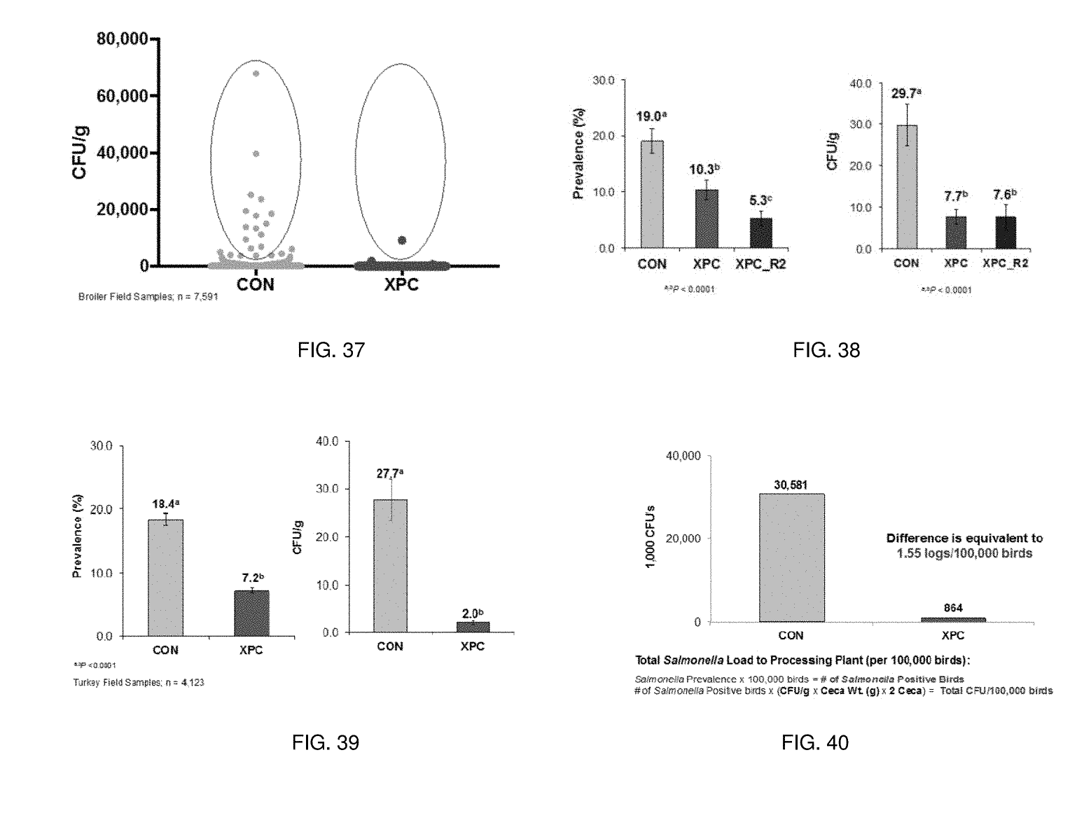

In the case broilers, the substantial reduction in average prevalence and numbers across commercial operations (FIG. 35), equated to a reduction, in Salmonella, of 1.71 log CFU/100,000 birds delivered at the processing plant (FIG. 36). Clearly (FIG. 37) XPC reduced total Salmonella load by reducing the number of birds with high concentrations of Salmonella on broiler farms. When a second generation of birds was introduced in to barns that previously contained birds fed XPC, prevalence of Salmonella on re-sampling was reduced further (FIG. 38), compared to prevalence in the preceding flock. This cumulative reduction in prevalence of Salmonella across generations of flocks likely reflects a reduced environmental load resulting from feeding XPC to the preceding flock.

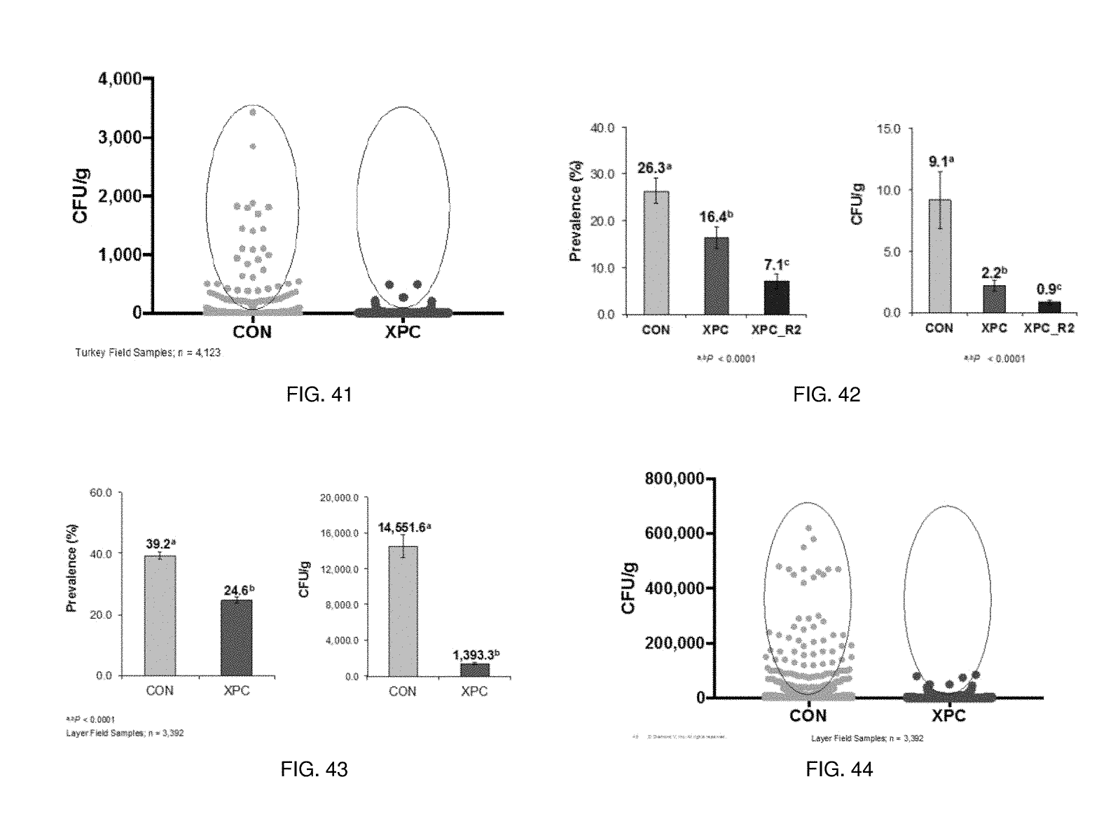

In the turkey flocks, the substantial reduction in prevalence and numbers (FIG. 39) across commercial operations, amounted to a reduction in Salmonella of 1.55 log CFU/100,000 birds delivered at the processing plant (FIG. 40). Clearly (FIG. 41) XPC reduced total Salmonella load by reducing the number of birds with high concentrations of Salmonella on farms. When a second generation of birds was introduced in to barns that previously contained birds fed XPC, prevalence and numbers of Salmonella on re-sampling were reduced further (FIG. 42) compared to the preceding flock. This cumulative reduction in prevalence and numbers of Salmonella across generations of flocks likely reflects a reduced environmental load resulting from feeding XPC to the preceding flock.

Looking specifically at layers, FIG. 43 illustrates that feeding XPC reduced prevalence and numbers of Salmonella, and as was the case for other bird species, this effect was largely the result of a reduced number of birds shedding high concentrations of Salmonella (FIG. 44) in XPC fed animals.

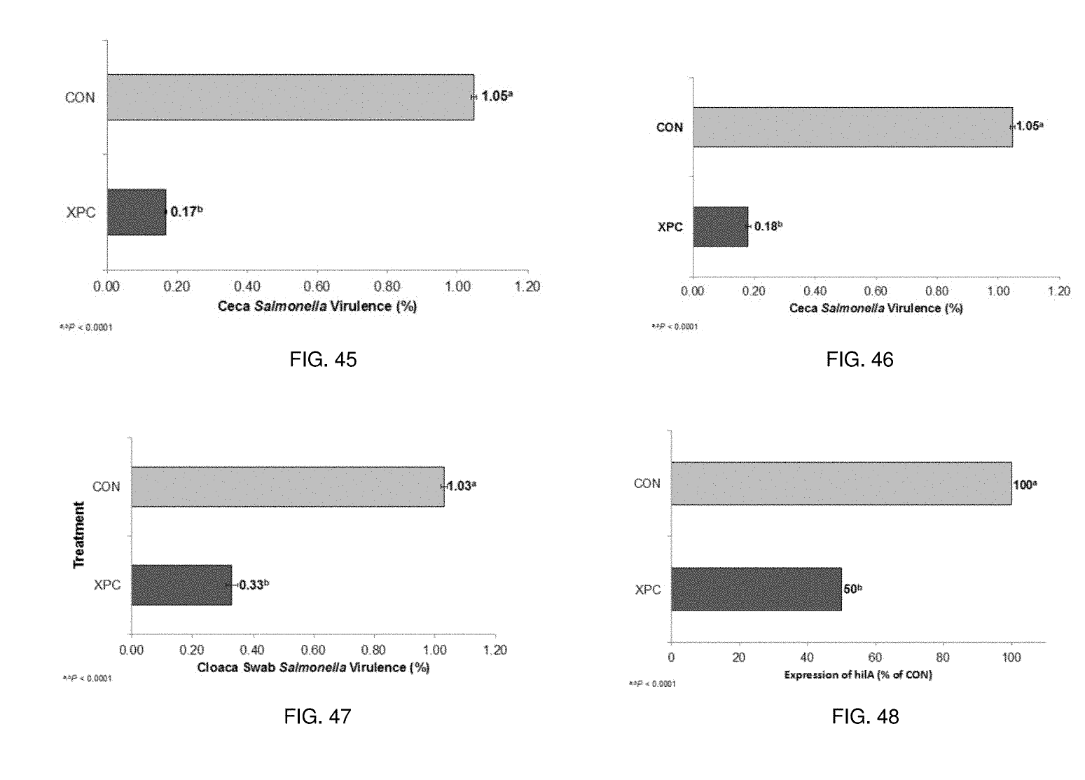

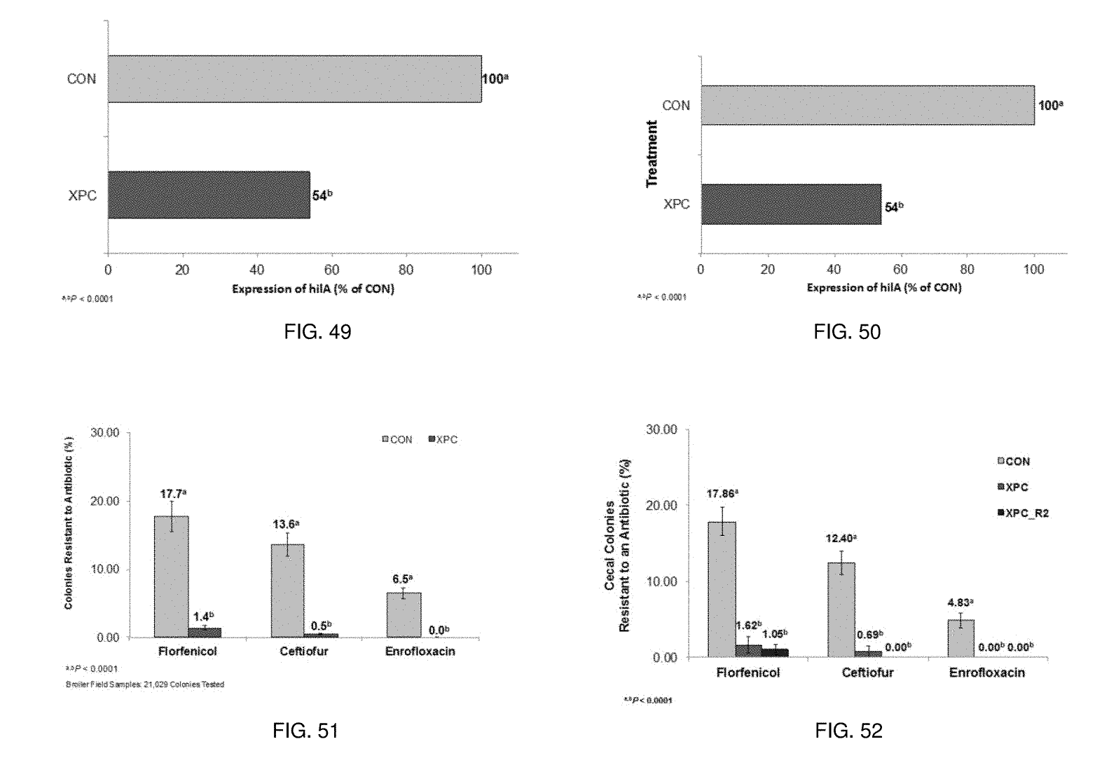

Feeding XPC reduced virulence of Salmonella recovered from broilers (FIG. 45), turkeys (FIG. 46) and layers (FIG. 47), and this reduction in virulence was associated with a reduced expression of the hilA gene in all 3 poultry species (FIGS. 48, 49 and 50, respectively).

Feeding XPC to broilers throughout their lifetime substantially reduced the level of resistance against Ceftiofur, Florfenicol and Enrofloxacin in wild Salmonella isolated from broiler ceca (FIG. 51). When a second generation of broilers was introduced in to barns that previously fed XPC, resistance of Salmonella against all 3 antibiotics was reduced further (FIG. 52) compared to the preceding flock. This cumulative reduction in antibiotic resistance of Salmonella across generations of flocks reflects a reduced environmental concentration resulting from feeding XPC to the preceding flock.

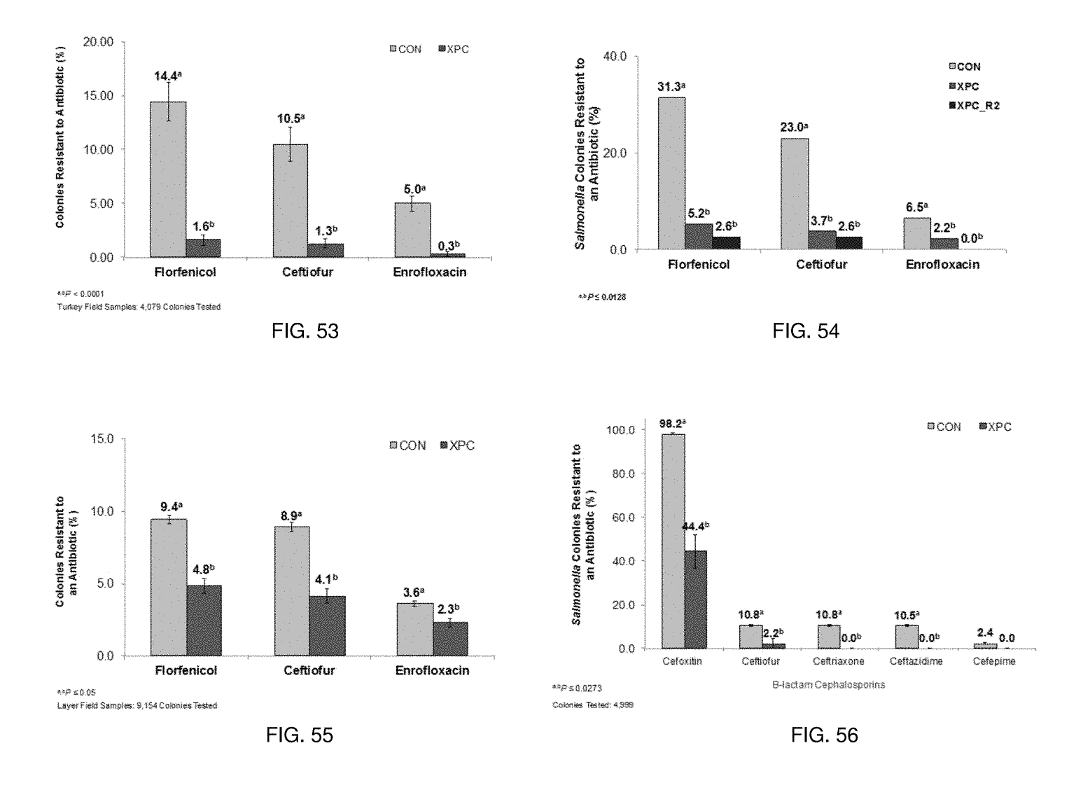

Feeding XPC to turkeys throughout their lifetime substantially reduced the level of resistance against Ceftiofur, Florfenicol and Enrofloxacin in wild Salmonella isolated from turkey ceca (FIG. 53). When a second generation of turkeys was introduced in to barns that previously contained fed XPC, resistance of Salmonella against all 3 antibiotics was reduced further (FIG. 54) compared to the preceding flock. This cumulative reduction in antibiotic resistance of Salmonella across generations of flocks reflects a reduced environmental concentration resulting from feeding XPC to the preceding flock.

Feeding XPC to laying hens throughout their lifetime substantially reduced the level of resistance against Ceftiofur, Florfenicol and Enrofloxacin in wild Salmonella isolated from the cloaca of these birds (FIG. 55). When a second generation of turkeys was introduced in to barns that previously contained fed XPC, resistance of Salmonella against all 3 antibiotics was reduced further (FIG. 54) compared to the preceding flock. This cumulative reduction in antibiotic resistance of Salmonella across generations of flocks reflects a reduced environmental concentration resulting from feeding XPC to the preceding flock.

In summary, feeding XPC reduced the virulence and resistance of Salmonella to Florfenicol, Ceftiofur and Enrofloxacin in all 3 species of poultry, under commercial conditions. Further, because feeding XPC reduced expression of the hilA gene in Salmonella, the reduced virulence of Salmonella was likely the result of reduced expression of the hilA gene.

### Example 6

### Effects of Feeding XPC, a Saccharomyces cerevisiae Fermentation Product on Antibiotic Resistance of Salmonella on Commercial Poultry Operations Using the Procedures of the National Antimicrobial Resistance Monitoring System

Field trials were performed on multiple broiler and layer farm operations, with matched houses/farms, to determine the effects of feeding XPC on antibiotic resistance of Salmonella isolated from the birds, against the full complement of antibiotics used by the National Antimicrobial Resistance Monitoring System (NARMS). The NARMS panel was established in 1996 by the Federal Drug Administration to monitor the antimicrobial therapies utilized in both veterinary and human medicine. The antimicrobial therapies included in the NARMS panel are (1) B-lactam Cephalosporins: Cefoxitin, Ceftiofur, Ceftriaxone, Ceftazidime, Cefepime; (2), Fluoroquinolones, Phenicols and Quinolones: Ciprofloxacin, Enrofloxacin, Florfenicol, Chloramphenicol, Nalidixic acid; (3) Aminoglycosides, B-lactam Monobactam, Penicillin: Gentamicin, Streptomycin, Aztronam, Ampicillin, Amox-cla, (4) Macrolides, Sulfonamides and Tetracycline: Azithromycin, Sulisoxazole, SMZ-TMP and Tetracycline.

On each farm, birds were fed the commercial diet in use at the specific farm, with (XPC; 1.25 kg/MT)) or without XPC (Control), from one day of age to market. The cloaca of layers were swabbed for microbial evaluation, and in the case of broilers, ceca were collected at the processing plant. Additionally, for layers, environmental samples were collected within each barn using established procedures known by those skilled in the art. All samples were processed and shipped overnight on ice. Salmonella antimicrobial minimal inhibitory concentrations (MICs) were determined by broth microdilution according to the Clinical and Laboratory Standards Institute (CLSI) standards), using a 96-well microtiter plate (Sensititre, Trek Diagnostic Systems, Thermo Fisher Scientific Inc., Cleveland, Ohio).

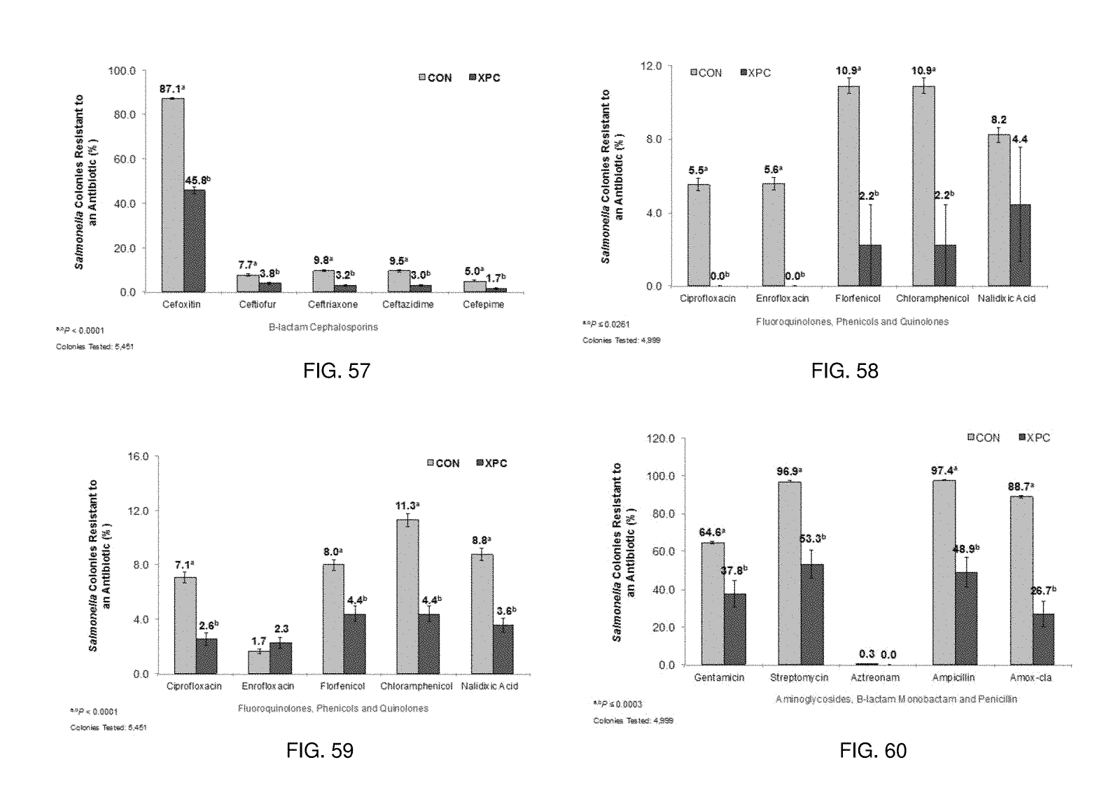

Feeding XPC to broilers or laying hens significantly (P<0.05) reduced resistance of Salmonella to all antibiotics classified as B-lactam Cephalosporin by between 40% and 100% (FIGS. 56 and 57, respectively) in the NARMS panel.

Feeding XPC to broilers or laying hens significantly (P<0.05) reduced resistance of Salmonella to all antibiotics classified as Fluoroquinolones, Phenicols and Quinolones (FIGS. 58 and 59, respectively) in the NARMS panel.

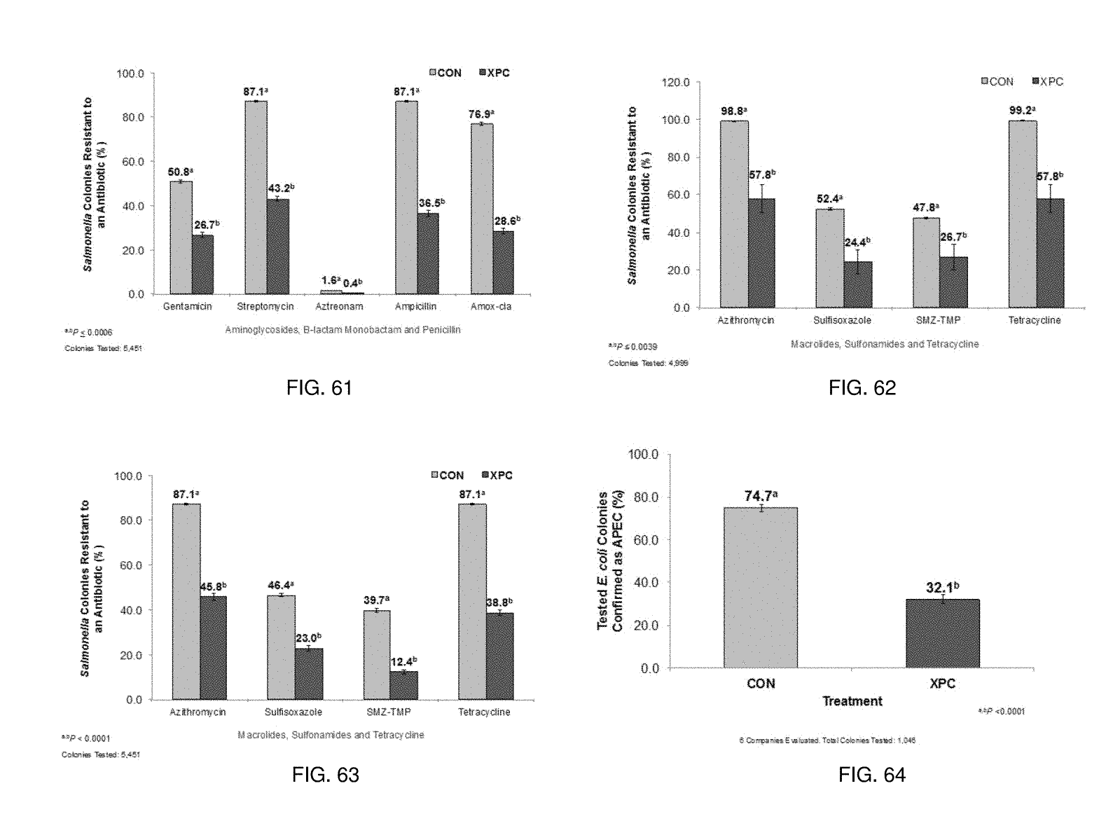

Feeding XPC to broilers or laying hens significantly (P<0.05) reduced resistance of Salmonella to all antibiotics classified as Aminoglycosides, B-lactam Monobactam, Penicillin, (FIGS. 60 and 61, respectively) in the NARMS panel.

Feeding XPC to broilers or laying hens significantly reduced resistance of Salmonella against all antibiotics classified as Macrolides, Sulfonamides and Tetracycline (FIGS. 62 and 63, respectively) in the NARMS panel.

In summary, feeding XPC to layers and broilers reduced the resistance of Salmonella isolated from these birds, to all 19 antimicrobial compounds contained in the NARMS panel. Because the antibiotic compounds on the NARMS panel represent several different mechanisms of antibiotic resistance, XPC had acts very broadly across antibiotic categories to restore sensitivity to antibiotics.

### Example 7

### Effects of Feeding XPC, a Saccharomyces cerevisiae Fermentation Product on Avian Pathogenic E. coli Prevalence, and Antibiotic Resistance of E. coli in Ceca Samples Taken from Commercial Broilers and Turkeys

Field trials were performed on multiple commercial broiler (6) and turkey (4) farm operations with paired houses/farms in order to determine the effects of feeding XPC on numbers and prevalence and virulence and antibiotic resistance of natural occurring Avian Pathogenic E. coli (APEC) in a commercial setting. On each farm, birds were fed the commercial diet in use at the specific farm, with (XPC; 1.25 kg/MT) or without XPC (Control), from one day of age to market age. Ceca were harvested at the processing plant, at 53 and 139 days of age for the broilers and turkeys respectively. All samples were shipped overnight on ice for E. coli analyses. 1. The proportion of E. coli that were APEC (APEC prevalence) were determined using the multiplex PCR methods of Johnson et al. (2008; J. Clin. Micro. 46(12):3987-3996). Isolated E. coli colonies were tested for resistance to Florfenicol, Ceftiofur and Enrofloxacin by microdilution, using standard methods known to those skilled in the art (CLSI 2017; Performance standards for antimicrobial disk and dilution susceptibility tests. Wayne, Pa.).

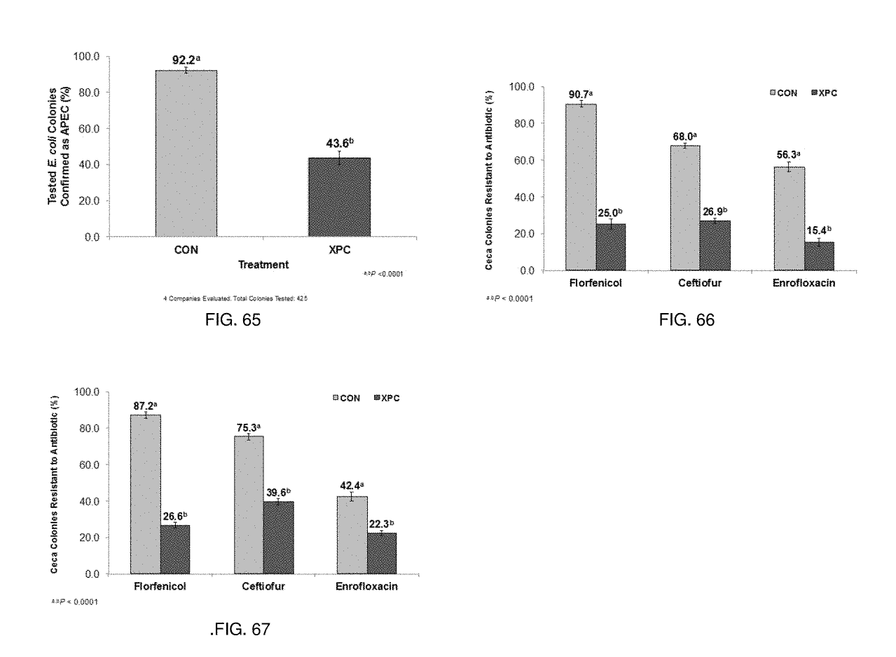

Approximately 75% (broilers, FIG. 64) and 92% (turkeys, FIG. 65) of E. coli isolated from ceca of birds fed control diets were APEC, feeding XPC more than halved the prevalence of APEC.

Feeding XPC to broilers (FIG. 66) and turkeys (FIG. 67) reduced resistance of E. coli to Florfenicol, Ceftiofur and Enrofloxacin.

In summary, resistance to the antibiotics Ceftiofur, Enrofloxacin and Florfenicol was reduced in the E. coli colonies isolated from birds fed XPC when compared to colonies isolated from birds fed a control diet.

Having thus described the invention in connection with the several embodiments thereof, it will be evident to those skilled in the art that various revisions can be made to the several embodiments described herein without departing from the spirit and scope of the invention. It is our intention, however, that all such revisions and modifications that are evident to those skilled in the art will be included with in the scope of the following claims. Any elements of any embodiments disclosed herein can be used in combination with any elements of other embodiments disclosed herein in any manner to create different embodiments.

Having described the invention, we claim:

1. A method for restoring the sensitivity of antibiotic resistant bacteria to antibiotics, comprising the steps of:
placing the antibiotic resistant bacterium into an environment that includes a fermented product;
wherein the fermented product is derived at least partially from Saccharomyces cerevisiae;
wherein the fermented product has a crude protein content, a crude fat content, and a crude fiber content;
the fermented product has ash and amino acids;
the fermented product has potassium, phosphorous and calcium.

2. The method of claim 1, wherein:
the antibiotic resistant bacteria is Salmonella.

3. The method of claim 1, wherein:
the antibiotic resistant bacteria is Escherichia coli.

4. The method of claim 3, wherein:
the Escherichia coli is Escherichia coli O157:H7.

5. The method of claim 1, wherein:
the antibiotic resistance bacteria is resistant to the antibiotic Florfenicol.

6. The method of claim 1, wherein:
the antibiotic resistance bacteria is resistant to the antibiotic Ceftiofur.

7. The method of claim 1, wherein:
the antibiotic resistance bacteria is resistant to the antibiotic Enrofloxacin.

8. The method of claim 1, further comprising the steps of:
expelling a SGI1 integron from the bacteria.

9. A method for reducing the expression of the hilA gene in bacteria, comprising the steps of:
placing the bacteria into an environment that includes a fermented product;
wherein the fermented product is derived at least partially from Saccharomyces cerevisiae;
wherein the fermented product has a crude protein content, a crude fat content, and a crude fiber content;
the fermented product has ash and amino acids;
the fermented product has potassium, phosphorous and calcium.

10. The method of claim 9, wherein:
the bacteria is Salmonella.

11. The method of claim 10, further comprising the step of:
expelling a SGI1 integron from the bacteria.

12. The method of claim 11, further comprising the steps of:
adding a fixed amount of the fermented product to feed for an animal;
feeding an animal the feed supplemented with the fermented product.

13. A method for restoring the sensitivity of antibiotic resistant bacteria to antibiotics, the bacteria located in an animal, comprising the steps of:
placing the bacteria into an environment that includes a fermented product;
wherein the fermented product is derived at least partially from Saccharomyces cerevisiae;
wherein the fermented product has a crude protein content, and a crude fat content;
the fermented product has ash and amino acids;
the fermented product has potassium, phosphorous and calcium;
feeding an animal the fermented product.

14. The method of claim 13, wherein:
the antibiotic resistance bacterium is resistant to the antibiotic Cefoxitin.

15. The method of claim 13, wherein:
the antibiotic resistance bacterium is resistant to the antibiotic Ceftriaxone.

16. The method of claim 13, wherein:
the antibiotic resistance bacterium is resistant to the antibiotic Ceftazidime.

17. The method of claim 13, further comprising the steps of:
a) Determining an average amount of feed consumed by the animal on a daily basis;
b) Determining an amount of a fermented product to supplement the average amount of feed the animal consumes on a daily basis;
c) Adding the amount of fermented product to the feed for a total feed amount.

18. The method of claim 17, wherein:
the amount of fermented product added to the feed corresponds to a range of 0.004% and 0.2% of the total feed.

19. The method of claim 17, wherein:
the amount of fermented product added to the feed is 0.125% of the total feed.

20. The method of claim 17, wherein:
the crude protein content is a minimum of 12.0%;
the crude fat content is a minimum of 1.2%;
the ash content is a maximum of 10.8%.
